SEQUENCE LISTING (15 sequences)

SEQ ID NO: 1 - protein, 37 aa, Artificial
  synthetic construct
HDEFERHAEGTFTSDVSSYLEGQAAKEFIAWLVKGRG

SEQ ID NO: 2 - protein, 31 aa, Artificial
  synthetic construct
HAEGTFTSDVSSYLEGQAAKEFIAWLVKGRG

SEQ ID NO: 3 - protein, 30 aa, Artificial
  synthetic construct
HAEGTFTSDVSSYLEGQAAKEFIAWLVKGR

SEQ ID NO: 4 - protein, 29 aa, Artificial
  synthetic construct
HAEGTFTSDVSSYLEGQAAKEFIAWLVKG

SEQ ID NO: 5 - protein, 28 aa, Artificial
  synthetic construct
HAEGTFTSDVSSYLEGQAAKEFIAWLVK

SEQ ID NO: 6 - protein, 27 aa, Artificial
  synthetic construct
HAEGTFTSDVSSYLEGQAAKEFIAWLV

SEQ ID NO: 7 - protein, 31 aa, Artificial
  synthetic construct
HVEGTFTSDVSSYLEGQAAKEFIAWLVKGRG

SEQ ID NO: 8 - protein, 31 aa, Artificial
  synthetic construct
HAQGTFTSDVSSYLEGQAAKEFIAWLVKGRG

SEQ ID NO: 9 - protein, 31 aa, Artificial; identical to SEQ ID NO: 8
  synthetic construct

SEQ ID NO: 10 - protein, 31 aa, Artificial
  synthetic construct
HAEGTFTSDVSKYLEGQAAKEFIAWLVKGRG

SEQ ID NO: 11 - protein, 31 aa, Artificial
  synthetic construct
HAEGTFTSDTSKYLEGQAAKEFIAWLVKGRG

SEQ ID NO: 12 - protein, 36 aa, Artificial
  synthetic construct
HDEFERHAEGTFTSDVSSYLEGQAAKEFIAWLVKGR

SEQ ID NO: 13 - protein, 31 aa, Artificial; identical to SEQ ID NO: 2
  synthetic construct

SEQ ID NO: 14 - protein, 31 aa, Artificial
  synthetic construct
XXEGTFTSDVSSYLXGQAAKXFIAWLVKGXX

SEQ ID NO: 15 - protein, 29 aa, Artificial
  synthetic construct
HAEGTFTSDVSSYLEGQAAKEFIAWLVKX

(19) United States
(12) Patent Application Publication
(10) Pub. No.: US 2009/0305964 A1
(43) Pub. Date: Dec. 10, 2009

(54) PHARMACEUTICAL PREPARATIONS OF A GLP-1 MOLECULE AND AN ANTI-EMETIC DRUG

(75) Inventors: Hans T. Schambye, Holte (DK); Henrik Nilsson, Copenhagen (DK); Christian Hansen, Vedbaek (DK)

Correspondence Address: BROWDY AND NEIMARK, P.L.L.C.;624 NINTH STREET, NW, SUITE 300, WASHINGTON, DC, 20001-5303, US

(73) Assignee: Gastrotech Pharma A/S, Copenhagen K (DK)

(21) Appl. No.: 11/912,051

(22) PCT Filed: Apr. 21, 2006
(86) PCT No.: PCT/DK2006/000211

§ 371 Date: Oct. 19, 2007

(30) Foreign Application Priority Data
Apr. 21, 2005 (DK) PA 2005 00583
Jun. 15, 2005 (DK) PA 2005 00879

(51) Int. Cl. A61K 38/22

(52) U.S. Cl. 514/12

(57) ABSTRACT

The present invention relates to a kit of parts comprising a GLP-1 molecule and an anti-emetic drug, said kit of parts being suitable for separate, sequential or/and simultaneous administration to a subject, preferably a human being. Also provided are combinations of GLP-1 or a GLP-1 analog with one or more anti-emetic drugs, as well as uses of the combinations in the manufacture of medicaments.

### FIELD OF INVENTION

The present invention relates to the combined therapeutic or prophylactic use of GLP-1 with anti-emetics to obtain a more effective and/or better tolerated treatment. This application is a non-provisional claiming priority from Danish patent applications No. PA 2005 00583 filed 21 Apr. 2005 and PA 2005 00879 filed 15 Jun. 2005, which are hereby incorporated by reference in their entirety. All patent and non-patent references cited in those applications, or in the present application, are also hereby incorporated by reference in their entirety.

### BACKGROUND OF INVENTION

### GLP-1

The development of peptide drugs for the treatment of various diseases has proven very successful. Peptide drugs include natural versions of peptide hormones as well as modified versions of these peptide hormones. One such peptide hormone is GLP-1.

The tissue distribution of GLP-1 has been investigated, with GLP-1 mRNA being detected in rat lung, pancreatic islets, stomach, kidney, hypothalamus and heart but not adipose, liver and skeletal muscle (Bullock et al., “Tissue distribution of messenger ribonucleic acid encoding the rat glucagon-like peptide-1 receptor”, Endocrinology. 1996 July; 137(7):2968-78).

Human GLP-1 (1-37) has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO:1). GLP-1 (1-37) is amidated post-translationally to yield GLP-1 (1-36)NH₂, which has the sequence His Asp Glu Phe Glu Arg His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO: 12); or is enzymatically processed to yield GLP-1 (7-37), which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg Gly (SEQ ID NO: 2). GLP-1 (7-37) can also be amidated to yield GLP-1 (7-36)amide, which together with GLP-1 (7-37) constitute the natural forms of the GLP-1 molecule, and which has the sequence His Ala Glu Gly Thr Phe Thr Ser Asp Val Ser Ser Tyr Leu Glu Gly Gln Ala Ala Lys Glu Phe Ile Ala Trp Leu Val Lys Gly Arg (NH₂) (SEQ ID NO:13). Likewise, GLP-1 (1-36)(NH₂) can be processed to GLP-1 (7-36)(NH₂).

The majority of circulating biologically active GLP-1 is found in the GLP-1 (7-36)amide form), with lesser amounts of the bioactive GLP-1 (7-37) form also detectable (Diabetes. 1994 April; 43(4):535-9). Both peptides appear equipotent in all biological paradigms studied to date (Diabetes. 1993 May; 42(5):658-61). GLP-1 is secreted from gut endocrine cells in response to nutrient ingestion and plays multiple roles in metabolic homeostasis following nutrient absorption.

An important locus for regulation of GLP-1 biological activity is the N-terminal degradation of the peptide by Dipeptidyl Peptidase (DPP-IV)-mediated cleavage at the position 2 alanine. Strategies designed to circumvent DPP-IV-mediated inactivation of GLP-1 include the generation of modified GLP-1 molecules engineered to exhibit DPP-IV-resistance. Several reports have documented the enhanced biological activity of such molecules in both normal and diabetic rodents. The lizard GLP-1-related peptide exendin-4 has a glycine in position 2, rendering the peptide resistant to DPP-IV-mediated degradation. This likely contributes significantly to the enhanced potency and stability of exendin-4 in vivo. (Diabetes. 1999 May; 48(5):1026-34).

The biological activities of GLP-1 include stimulation of glucose-dependent insulin secretion and insulin biosynthesis, inhibition of glucagon secretion and gastric emptying, and inhibition of food intake. GLP-1 appears to have a number of additional effects in the GI tract and central nervous system (Diabetes 1998 47(2):159-69, Endocrinology. 2001 February; 142(2):521-7, Curr Pharm Des. 2001 September; 7(14):1399-412, Gastroenterology. 2002 February; 122(2):531-44).

In studies using rat parietal cell preparations, both exendin-4 and GLP-1 display similar properties with respect to H+ and cAMP production and the actions of these peptides are blocked by the GLP-1 receptor antagonist exendin(9-39) (Eur J. Pharmacol. 1994 Oct. 14; 269(2):183-91.

### SUMMARY OF INVENTION

The invention relates to products, uses and methods involving GLP-1 or a GLP-1 analog and an anti-emetic drug. Thus, in one aspect of the present invention is provided a combination of GLP-1 and/or a GLP-1 analog, and an anti-emetic drug, such as any of the anti-emetic drugs disclosed herein.

Another aspect of the invention relates to a kit of parts comprising:
i. GLP-1 or a GLP-1 analog and
ii. an anti-emetic drug
   for separate, sequential and/or simultaneous administration to a subject in need of treatment and/or for use in therapy.

A further aspect relates to a product comprising
i. GLP-1 or a GLP-1 analog and
ii. an anti-emetic drug
   in a combined medicament and/or in two distinct medicaments for separate, sequential or/and simultaneous administration to a subject in need of treatment.

Another aspect of the invention relates to the use of a composition comprising GLP-1 or a GLP-1 analog and an anti-emetic drug for the manufacturing of a medicament for the treatment, prevention or prophylaxis of a disease or disorder amenable to treatment with said peptide drug.

In a further aspect, the invention relates to the use of GLP-1 or a GLP-1 analog and/or an anti-emetic drug for the manufacture of (a) medicament(s) for use in combination therapy aimed at the treatment, prophylaxis and/or prevention of a disease or disorder amenable to treatment with said peptide drug; and involving separate, sequential and/or simultaneous administration of:
i. a medicament comprising both GLP-1 or a GLP-1 analog and an anti-emetic drug or,
ii. a medicament comprising GLP-1 or a GLP-1 analog and a medicament comprising an anti-emetic drug or,
iii. a medicament comprising both GLP-1 or a GLP-1 analog and an anti-emetic drug, and a separate medicament comprising GLP-1 or a GLP-1 analog or,
iv. a medicament comprising both GLP-1 or a GLP-1 analog and an anti-emetic drug and a separate medicament comprising an anti-emetic drug.

In a further aspect, the invention relates a method of treatment, prophylaxis or prevention of a disease or disorder comprising administering to a subject one or more medicaments comprising,
i. GLP-1 or a GLP-1 analog and
ii. an anti-emetic drug
   separately, sequentially or/and simultaneously, wherein the disease or disorder is selected from the group consisting of obesity, diabetes, hypertension, metabolic syndrome, or for the modulation of blood glucose level.

Further diseases included are functional gastrointestinal disorders, such as irritable bowel syndrome and functional dyspepsia.

### Description of Sequence Listing

Sequence ID NO: 1: amino acid sequence of Human GLP-1 (1-37)

Sequence ID NO: 2: amino acid sequence of Human GLP-1 (7-37)

Sequence ID NO: 3: amino acid sequence of Human GLP-1 (7-36)

Sequence ID NO: 4: amino acid sequence of Human GLP-1 (7-35)

Sequence ID NO: 5: amino acid sequence of Human GLP-1 (7-34)

Sequence ID NO: 6: amino acid sequence of Human GLP-1 (7-33)

Sequence ID NO: 7: amino acid sequence of Human Val8-GLP-1 (7-37)

Sequence ID NO: 8: amino acid sequence of Human Gln9-GLP-1 (7-37)

Sequence ID NO: 9: amino acid sequence of Human D-Gln9-GLP-1 (7-37)

Sequence ID NO: 10: amino acid sequence of Human Lys18-GLP-1 (7-37)

Sequence ID NO: 11: amino acid sequence of Human Thr16-Lys18-GLP-1 (7-37)

Sequence ID NO: 12: amino acid sequence of Human GLP-1 (1-36)NH₂

Sequence ID NO: 13: amino acid sequence of Human GLP-1 (7-36)amide

Sequence ID NO: 14: amino acid sequence of a preferred GLP-1 analog

Sequence ID NO: 15: amino acid sequence of a preferred GLP-1 analog

### DETAILED DESCRIPTION OF THE INVENTION

### Definitions

Affinity: the strength of binding between receptors and their ligands, for example between an antibody and its antigen.

Amino Acid Residue: An amino acid formed upon chemical digestion (hydrolysis) of a polypeptide at its peptide linkages. The amino acid residues described herein are preferably in the “L” isomeric form. However, the amino acid encompasses every amino acid such as L-amino acid, D-amino acid, alpha-amino acid, beta-amino acid, gamma-amino acid, natural amino acid and synthetic amino acid or the like as long as the desired functional property is retained by the polypeptide. Further included are natural or synthetic amino acids which have been modified. NH2 refers to the free amino group present at the amino terminus of a polypeptide. COOH refers to the free carboxy group present at the carboxy terminus of a polypeptide, Standard polypeptide abbreviations for amino acid residues are used herein.

It should be noted that all amino acid residue sequences represented herein by formulae have a left-to-right orientation in the conventional direction of amino terminus to carboxy terminus. Furthermore, it should be noted that a dash at the beginning or end of an amino acid residue sequence indicates a peptide bond to a further sequence of one or more amino acid residues or a covalent bond to an amino-terminal group such as NH₂ or acetyl or to a carboxy-terminal group such as COOH.

Concentration equivalent: A concentration equivalent is an Equivalents dosage being e.g. defined as the dosage of a GLP-1-like compound having in vitro and/or in vivo the same response as evaluated from a dosage-response curve of wild-type GLP-1.

Dissociation constant, Kd: a measure to describe the strength of binding (or affinity or avidity) between receptors and their ligands, for example a GLP-1 molecule and the GLP-1 receptor. The smaller Kd, the stronger binding.

GLP-1 analog: by the term GLP-1 analog is meant any molecule capable of binding to and activating the GLP-1 receptor, such as e.g. any of the molecules disclosed in the section entitled “GLP-1 molecule” below. The terms “GLP-1 molecule” and “GLP-1 analog” are preferably used interchangeably, i.e. by “GLP-1 molecule” is meant e.g. GLP-1 in its natural forms, a GLP-1 analog or a GLP-1 derivative.

Individual: A living animal. In preferred embodiments, the subject is a mammal, including humans and non-human mammals such as dogs, cats, pigs, cows, sheep, goats, horses, rats, and mice. In the most preferred embodiment, the subject is a human.

Isolated: is used to describe the various GLP-1 molecules, polypeptides and nucleotides disclosed herein, that have been identified and separated and/or recovered from a component of its natural environment or of its production process. Contaminant components of its natural environment are materials that would typically interfere with diagnostic or therapeutic uses for the polypeptide, and may include enzymes, hormones, and other proteinaceous or non-proteinaceous solutes. In preferred embodiments, the polypeptide will be purified.

Modified amino acid: an amino acid wherein an arbitrary group thereof is chemically modified. One, two, three or more modified amino acids can be used in any of the GLP-1 molecules disclosed herein. In particular, a modified amino acid chemically modified at the alpha-carbon atom in an alpha-amino acid is preferable.

Polypeptide: The phrase polypeptide refers to a molecule comprising amino acid residues which do not contain linkages other than amide linkages between adjacent amino acid residues.

### Indications

It is contemplated that therapeutic treatments using GLP-1 or a GLP-1 analog can be greatly improved by the co-administration of an anti-emetic drug, whereby unwanted side-effects of the GLP-1 or GLP-1 analog are diminished and the treatment thereby improved. Also, the negative consequences of these side effects, e.g. reduced food intake and/or nutrient utilization, may be avoided and thereby contribute to the efficacy of the treatment. By avoiding or reducing the side effect it is contemplated that the GLP-1 or GLP-1 analog can be used in a higher dosage or be continued for a longer period of time (thereby improving the efficacy of the treatment) and thus providing a more effective treatment of the disease or disorder in question. The patient may avoid premature termination of a GLP-1 or GLP-1 analog treatment or reduction of the GLP-1 or GLP-1 analog dose, and may therefore experience faster recovery including prior reestablishment of normal life, increased quality of life. The patient may further end medication earlier due to increased physical well being and nutritional state.

### Kit of Parts

The advantage of the invention is obtained by coordinated administration of at least GLP-1 or a GLP-1 analog and an anti-emetic drug which may be administered as a separate, sequential and/or simultaneous administration to a subject in need of treatment. The subject in need of treatment is preferably a patient which suffers from or is in risk of suffering from a disease or disorder treatable with the GLP-1 or GLP-1 analog, and thus is subjected to preventive, prophylactic or therapeutic treatment with the relevant GLP-1 or GLP-1 analog, which patient furthermore is in risk of or suffers from nausea or emesis or a similar condition resulting from the treatment with the peptide drug. It will be understood that the level of nausea or emesis experienced as a side effect from treatment with GLP-1 or a GLP-1 analog may vary considerably from patient to patient. Therefore, the treatment according to the invention is usually preferably adapted to the individual patient with the aim of obtaining maximum efficiency and tolerability of the GLP-1 and GLP-1 analog treatment, while at the same time reduce or remove nausea or emesis experienced from the patient in peptide drug treatment.

While the peptide drug and the anti-emetic drug may be provided as to separate drugs, it may be an advantage to provide said drugs in the form of a single product, i.e. a kit of parts.

Accordingly, in one aspect the invention relates to a kit of parts comprising
i. GLP-1 or a GLP-1 analog and
ii. an anti-emetic drug
   for separate, sequential and/or simultaneous administration to a subject in need of treatment.

The kit of parts may comprise a combined medicament comprising an effective amount of the GLP-1 or GLP-1 analog and an effective amount of the anti-emetic drug.

Alternatively, in the kit of parts the GLP-1 or GLP-1 analog and the anti-emetic drug may be provided as distinct medicaments.

Furthermore, the kit of parts may comprise

a) a combined medicament comprising:
i. GLP-1 or a GLP-1 analog and an anti-emetic drug and
   b) one or more discrete medicament(s) comprising:
i. GLP-1 or a GLP-1 analog and/or
ii. an anti-emetic drug.
   for separate, sequential or/and simultaneous administration to a subject in need of treatment.

By combining two or more different medicaments in one package the kit of parts may be suited for treatment wherein the dosage regime may vary during the treatment (see below). The combined and/or distinct medicament(s) preferably comprise(s) the relevant amounts of the respective drugs formulated so as to be released in the relevant body compartment at the relevant time for each drug (e.g. simultaneously, sequentially or separately).

Preferably, the kit of parts provides each of the GLP-1 or GLP-1 analog and the anti-emetic drug in a respective dosage form suitable for and effective in the therapeutic treatment in question.

The dosage form contains a sufficient amount of active compound such that a desirable effect can be obtained when administered to a subject. Thus, it is preferred that the medical packaging comprises a number of dosage units corresponding to the relevant dosage regimen.

Furthermore, the kit of parts may provide for the respective drugs to be administered in either single or multiple doses.

The kit of parts is preferably prescribed for use in the prevention, prophylaxis or treatment of a condition or disease for which the GLP-1 or GLP-1 analog is therapeutically effective, the anti-emetic drug of the kit of parts serving to reduce or remove an undesirable side effect (such as nausea and emesis) resulting from therapeutic treatment with the GLP-1 or GLP-1 analog.

Preferably, a kit contains instructions indicating the use of the dosage form to achieve a desirable effect and the amount of dosage form to be taken over a specified time period. Accordingly, in one embodiment the kit of parts comprises instructions for administering the pharmaceutical composition.

The GLP-1 or GLP-1 analog and the anti-emetic drug to be comprised in the kit of parts of the invention may be any of those described herein, for example below in the sections entitled “GLP-1 or GLP-1 analog” and “anti-emetic drug”, respectively.

### Peptide Drugs

Naturally occurring peptides include peptides found in fungi, plants, animals, mammals, lizard and humans. The peptides to be used in the present invention may be derived from any species, such as goat, horse, cow, pig, monkey, rat, mouse, rabbit and bird. Peptides derived from Homo sapiens are preferred. The peptide drugs may be synthetic molecules having a substantially similar or higher binding affinity to the receptor than the naturally occurring peptide hormone known to bind the receptor. Such molecules may be termed analogs as they are capable of performing substantially the same function as the peptide hormone.

In the present context, the term peptide hormone is used to indicate the naturally occurring hormone as well as an analog or homologue thereof which has a pharmaceutically relevant activity, e.g. the same as its parent peptide.

Analogs and homologues may be developed by a person skilled in the art, e.g. based on the knowledge of the peptide hormone in question and the receptor(s) affected by a peptide hormone using in vitro receptor binding assays.

In a preferred embodiment the homologue has an amino acid sequence which is at least 60% identical to the peptide hormone. More preferably the homologue is at least 65%, such as at least 70% identical, such as at least 75% identical, such as at least 80% identical, such as at least 85% identical, such as at least 90% identical, such as at least 95%, such as at least 96% such as at least 97% such as at least 98% identical or such as at least 99% identical to the peptide hormone.

The percentages mentioned above relate to the identity of the sequence of a homologue as compared to the peptide hormone as may be obtained using suitable alignment programs including the algorithm GAP, BESTFIT, or FASTA in the Wisconsin Genetics Software Package Release 7.0, using default gap weights.

### GLP-1 and Analogs

The present invention relates to Glucagon-like peptide 1 (GLP-1) and analogs thereof. This includes GLP-1 molecules from any species and in particular Homo sapiens. GLP-1 exists in several processed forms as GLP-1 (1-37) (SEQ ID NO: 1) is N-terminally truncated by posttranslational processing in the intestinal L cells resulting in GLP-1 (7-37) (SEQ ID NO: 2) and GLP-1 (7-36)-amide. The C-terminal amidation is neither important for the metabolism of GLP-1 nor for its effects on the endocrine pancreas. GLP-1 usually referrers to GLP-1 (7-36) (SEQ ID NO: 3). Therefore the amino-terminus of GLP-1 (7-36) has been assigned number 7 and the carboxy-terminus, number 36. The amino acid sequence of human GLP-1 (7-36) is identified by SEQ ID NO: 3. Biologically active molecules include the peptides, GLP-1 (7-37), GLP-1 (7-36), GLP-1 (7-35), GLP-1 (7-34) and GLP-1 (7-33).

Preferred GLP-1 peptides are GLP7-34, GLP7-35, and also GLP7-36 which are disclosed in U.S. Pat. No. 5,118,666. These compounds are the biologically processed forms of GLP-1 having insulinotropic properties. Native GLP-1 (7-36) is rapidly degraded to the biologically inactive GLP-1 (9-36) by DPP-IV (N-terminal degradation) in vivo in the circulation, see e.g. Meier et al, Glucagon-like peptide 1 as a regulator of food intake and body weight: therapeutic perspectives. Eur. J. Pharmacol. 440 (2002): 269-279.

Numerous homologues, analogs and derivates of GLP-1, including agonists of the GLP-1 receptor, agonists of the GLP-1 signal transduction cascade, compounds that stimulate synthesis of endogenous GLP-1, compounds that stimulate release of endogenous GLP-1, and pharmaceutically-acceptable salts thereof have been described, see e.g. WO03018516, WO9808871, WO9943706, U.S. Pat. No. 5,118,666, U.S. Pat. No. 5,545,618 and US20040018975. In the present application, such homologues, analogs and derivatives are termed GLP-1 analogs.

The GLP-1 molecule to be used in the present invention may, e.g., be an analog of one of the active GLP-1 peptides; GLP-1 (7-37), GLP-1 (7-36), GLP-1 (7-35), GLP-1 (7-34) and GLP-1 (7-33) including one or more amino acid substitutions and/or a truncation of one or more amino acid residues at the C-terminus and/or contain. Analogs comprising one or more amino acid substitutions located in position 7, 8, 9 or 10 are preferred. Analogs having D-amino acid substitutions in position 7 and/or 8 and/or N-alkylated or N-acylated amino acids in position 7 have been found to be particularly resistant to degradation in vivo.

Analogs of GLP-1, known in the art include, for example, Val.8-GLP7-37 (SEQ ID NO: 7), Gln9-GLP7-37 (SEQ ID NO: 8), D-Gln9-GLP7-37 (SEQ ID NO: 9), Lys18-GLP7-37 (SEQ ID NO: 10) and Thr16-Lys18-GLP7-37 (SEQ ID NO: 11). Other GLP-1 analogs are disclosed in U.S. Pat. Nos. 5,545,618, 5,545,618 and patent application US20040018975.

The following GLP-1 molecules have been or are in pre-clinical or clinical development:

Native GLP-1 for the treatment of type 2 diabetes (See: Effect of 6-week course of glucagon-like peptide 1 on glycaemic control, insulin sensitivity, and beta-cell function in type 2 diabetes: a parallel-group study. Lancet. 2002 Mar. 9; 359(9309):824-30); native GLP-1 for the treatment of elderly subjects (See Effects of 3 Months of Continuous Subcutaneous Administration of Glucagon-Like Peptide 1 in Elderly Patients With Type 2 Diabetes. Diabetes Care. 2003 October; 26(10):2835-2841);

In a preferred embodiment the peptide drug is native GLP-1 (7-36).

NN2211 (also termed Liraglutide), is an acylated albumin-bound human GLP-1 analog for which phase II clinical data have been reported;

CJC-1131 is based on GLP-1-albumin drug affinity complex (DAC) technology and is a GLP-1 analogue engineered for covalent coupling to albumin. The GLP-1 albumin compound retains the ability to bind to and activate the GLP-1 receptor, and appears to exhibit highly similar biological actions, compared to native GLP-1, when assessed using cells in vitro, or rodents in vivo. For an overview of CJC-1131, see Development and characterization of a glucagon-like Peptide 1-albumin conjugate: the ability to activate the glucagon-like Peptide 1 receptor in vivo. Diabetes. 2003 March; 52(3):751-9.

BIM51077 is a human GLP-1 derivative, originally developed and taken through Phase 1 studies by Ipsen, now licensed to Roche.

LY315902 is a Lilly DPP-IV-resistant GLP-1 analogue and LY307161 SR is a sustained release formulation of a GLP-1 analog suitable for once daily administration.

LY307161 efficacy has been examined in human clinical trials.

GTP-010 is a GLP-1 analog of Lilly and Gastrotech.

Further analogs include AC2592 (GLP-1) and DAT™:GLP-1 developed by Amylin and Conjuchem respectively.

Exendin-4 (also termed Exenatide), is a naturally occurring DPP-IV-resistant GLP-1 analog which is in clinical development for the treatment of diabetes. Eli Lilly/Amylin have filed a new drug application (NDA) with the US food and drug administration (FDA) for exendin-4. Further included is Exenatide LAR developed by Lilly/Amylin and Alkermes.

ZP10 is an exendin-4 derivative being developed by Zealand Pharmaceuticals for the treatment of diabetes.

It is contemplated that each of the above mentioned GLP-1 molecules are candidate peptide drugs for use in the present invention.

In further embodiments the GLP-1 analog is selected from the group consisting of: NN2211 (liraglutide), CJC-1131, BIM51077, LY315902, LY307161, GTP-010, AVE-10 (ZP10), AC2592 (GLP-1), DAT™:GLP-1 Exendin-4, Exenatide LAR and ZP10.

One preferred GLP-1 analog useful in the present invention is a homologue with at least 80% sequence identity to GLP-1 (7-36)amide or GLP-1 (7-37), such as at least 90% sequence identity to GLP-1 (7-36)amide or GLP-1 (7-37), for example at least 95% sequence identity to GLP-1 (7-36)amide or GLP-1 (7-37).

### GLP-1 Molecule

The present invention relates to the use of GLP-1 molecule(s). The term “GLP-1 molecule” is used herein to refer to any molecule capable of binding to and activating the GLP-1 receptor. Methods for assaying the functional activity of the GLP-1 molecules for use in the present invention are described below in the section entitled “Functional activity of GLP-1 molecule”. It is to be understood that the activity of the GLP-1 molecules for use in the present invention can be less potent or more potent than native GLP-1 (7-36)amide.

Preferably, the GLP-1 molecule for use in the present invention is a polypeptide. One preferred group of compounds with this activity are the GLP-1 polypeptides originally sequenced from natural sources. Thus, a preferred molecule for use in the present invention may be selected from the group consisting of: GLP-1 (7-36)amide, and GLP-1 (7-37).

The present invention further includes the use of recombinant or synthetically produced human GLP-1 peptides as well as GLP-1 peptides derived from other species, whether recombinant or synthetic. Thus in one embodiment, the GLP-1 molecule is a homologue of GLP-1.

In one preferred embodiment of the present invention, the GLP-1 molecule is a GLP-1 molecule having one or more (such as 15 or fewer, for example 13 or fewer, such as 11 or fewer, for example 9 or fewer, such as 7 or fewer, for example 5 or fewer, such as 3 or fewer, for example 2 or fewer, such as 1 or fewer) amino acid substitutions, deletions, inversions, or additions relative to GLP-1 (7-37) and may include the D-amino acid forms. Numerous GLP-1 analogs are known in the art and include, but are not limited to, GLP-1 (7-34), GLP-1 (7-35), GLP-1 (7-36) NH2, Gln9-GLP-1 (7-37), d-Gln9-GLP1 (7-37), Thr16-Lys18-GLP-1 (7-37), and Lys18-GLP-1 (7-37), Gly′-GLP-1 (7-36) NH2, Gly′-GLP1 (7-37) OH, Val′-GLP-1 (7-37) OH, Met8-GLP-1 (7-37) OH, acetyl-Lys9-GLP-1 (7-37), Thr9GLP-1 (7-37), D-Thr9-GLP-1 (7-37), Asn9-GLP-1 (7-37), D-Asn9-GLP-1 (7-37), Ser22-Arg23 Arg24-Gln26-GLP-1 (7-37), Arg23-GLP-1 (7-37), Arg24-GLP-1 (7-37), a-methyl-Ala8-GLP-1 (736) NH2, and Gly′-Gln2′-GLP-1 (7-37) OH, and the like.

Other GLP-1 analogs consistent with the present invention are described by the formula:

(SEQ ID NO: 14)

R1-X-Glu-Gly-Thr-Phe-Thr-Ser-Asp-Val-Ser-Ser-Tyr-
Leu-Y-Gly-Gln-Ala-Ala
Lys-Z-Phe-Ile-Ala -Trp-Leu-Val-Lys-Gly-Arg-R2
wherein:

R1 is selected from the group consisting of L-histidine, D-histidine, desaminohistidine, 2-amino-histidine, beta-hydroxy-histidine, homohistidine, alpha-fluoromethylhistidine, and alpha-methyl-histidine;
X is selected from the group consisting of Ala, Gly, Val, Thr, Ile, and alpha-methyl-Ala;
Y is selected from the group consisting of Glu, Gln, Ala, Thr, Ser, and Gly;
Z is selected from the group consisting of Glu, Gln, Ala, Thr, Ser, and Gly; and R2 is selected from the group consisting of NH2, and Gly-OH.

GLP-1 analogs also have been described in WO 91/11457, and include GLP-1 (7-34), GLP-1 (7-35), GLP-1 (7-36), or GLP-1 (7-37), or the amide form thereof, and pharmaceutically-acceptable salts thereof, having at least one modification selected from the group consisting of:

(a) substitution of glycine, serine, cysteine, threonine, asparagine, glutamine, tyrosine, alanine, valine, isoleucine, leucine, methionine, phenylalanine, arginine, or D-lysine for lysine at position 26 and/or position 34; or substitution of glycine, serine, cysteine, threonine, asparagine, glutamine, tyrosine, alanine, valine, isoleucine, leucine, methionine, phenylalanine, lysine, or a D-arginine for arginine at position 36;
(b) substitution of an oxidation-resistant amino acid for tryptophan at position 31;
(c) substitution of at least one of: tyrosine for valine at position 16; lysine for serine at position 18; aspartic acid for glutamic acid at position 21; serine for glycine at position 22; arginine for glutamine at position 23; arginine for alanine at position 24; and glutamine for lysine at position 26; and
(d) substitution of at least one of: glycine, serine, or cysteine for alanine at position 8; aspartic acid, glycine, serine, cysteine, threonine, asparagine, glutamine, tyrosine, alanine, valine, isoleucine, leucine, methionine, or phenylalanine for glutamic acid at position 9; serine, cysteine, threonine, asparagine, glutamine, tyrosine, alanine, valine, isoleucine, leucine, methionine, or phenylalanine for glycine at position 10; and glutamic acid for aspartic acid at position 15; and
(e) substitution of glycine, serine, cysteine, threonine, asparagine, glutamine, tyrosine, alanine, valine, isoleucine, leucine, methionine, or phenylalanine, or the D- or N-acylated or alkylated form of histidine for histidine at position 7; wherein, in the substitutions described in (a), (b), (d), and (e), the substituted amino acids can optionally be in the D-form and the amino acids substituted at position 7 can optionally be in the N-acylated or N-alkylated form.

Preferred GLP-1 molecules used in the present invention also include analogs of GLP-1 (7-37) NH2 and GLP-1 (7-37) in which one or more amino acids which are not present in the original sequence are added or deleted, and derivatives thereof.

Specifically, His and desamino-histidine are preferred for R and/or Ala, Gly and Val are preferred at the “X” position. Also, Glu and Gln are preferred for at the “Y” position. Glu and Gln are preferred at the “Z” position and Gly-OH is preferred for R2.

A particularly preferred GLP-1 analog is known as Val (8) GLP-1 (V8GLP-1) and has a formula according to SEQ ID NO: 14 (see the previous page), wherein R, is L-histidine, X is Val, Y is Glu, Z is Glu and R2 is Gly-OH.

In another preferred embodiment of the present invention, the GLP-1 molecule is a GLP-1 derivative. A “GLP-1 derivative” is defined as a molecule having the amino acid sequence of GLP-1 (7-37) or of a GLP-1 analog, but additionally comprises chemical modification of one or more of its amino acid side groups, a-carbon atoms, terminal amino group, or terminal carboxylic acid group. A chemical modification includes, but is not limited to, adding chemical moieties, creating new bonds, and removing chemical moieties. Modifications at amino acid side groups include, without limitation, acylation of lysine s-amino groups, N alkylation of arginine, histidine, or lysine, alkylation of glutamic or aspartic carboxylic acid groups, and deamidution of glutamine or asparagine. Modifications of the terminal amino include, without limitation, the des-amino, N-lower alkyl, N-di-lower alkyl, and N-acyl modifications. Modifications of the terminal carboxy group include, without limitation, the amide, lower alkyl amide, dialkyl amide, and lower alkyl ester modifications. Lower alkyl is C1-C4 alkyl. Furthermore, one or more side groups, or terminal groups, may be protected by protective groups known to the ordinarily-skilled protein chemist. The a-carbon of an amino acid may be mono- or dimethylated.

Other GLP-1 derivatives include molecules which are selected from the group consisting of a peptide having the amino acid sequence:

(SEQ ID NO: 15)

NH2-His′-Ala-Glu-Glyl-Thr-Phe-Thr-Ser-Asp-Val-Ser-
Ser-Tyr-Leu-Glu-Gly Gln-Ala-Ala-Lys-Glu-Phe-Ile-
Ala-Trp-Leu-Val-X
and

pharmaceutically-acceptable salts thereof, wherein X is selected from the group consisting of:—
Lys and Lys-Gly; and a derivative of said peptide, wherein said peptide is selected from the group consisting of: a pharmaceutically-acceptable lower alkylester of said peptide; and a pharmaceutically-acceptable amide of said peptide, selected from the group consisting of amide, lower alkyl amide, and lower dialkyl amide.

Yet other GLP-1 derivatives appropriate for use in the present invention include compounds claimed in U.S. Pat. No. 5,512,549 described by the formula:

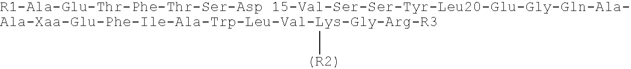

wherein R1 is selected from the group consisting of 4-imidazopropionyl, 4-imidazoacetyl, or 4-imidazo-a, a dimethyl-acetyl; R2 is selected from the group consisting of C6 Clo unbranched acyl, or is absent; R3 is selected from the group consisting of Gly-OH or NH2; and, Xaa is Lys or Arg, may be used in present invention.

Further preferred GLP-1 molecules capable of activating the GLP-1 receptor and suitable for use in the present invention include, but are not restricted to, any of the following molecules, which have previously been demonstrated to be capable of activating the GLP-1 receptor:
GLP-1 (7-36)amide
GLP-1 (7-37)
exendin-4
GLP-1 (7-34)
GLP-1 (7-35)
GLP-1 (7-36)
Gln9-GLP-1 (7-37)
D-Gln9-GLP-1 (7-37)
Thr16-Lys18-GLP-1 (7-37)
Lys18-GLP-1 (7-37)
GLP-1 (7-34)
GLP-1 (7-35)
GLP-1 (7-36)
GLP-1 (7-36)NH2
Gly8-GLP-1 (7-36)NH2
Gln9-GLP-1 (7-37)
D-Gln9-GLP-1 (7-37)
acetyl-Lys9-GLP-1 (7-37)
Thr9-GLP-1 (7-37)
D-Thr9-GLP-1 (7-37)
Asn9-GLP-1 (7-37)
D-Asn9-GLP-1 (7-37)
Ser22-Arg23-Arg24-Gln26-GLP-1 (7-37)
Thr16-Lys18-GLP-1 (7-37)
Lys18-GLP-1 (7-37)
Arg23-GLP-1 (7-37)
Arg24-GLP-1 (7-37)
GLP-1 (7-34)
GLP-1 (7-35)
GLP-1 (7-36) NH2
Gln9-GLP-1 (7-37)
d-Gln9-GLP1 (7-37)
Thr16-Lys18-GLP-1 (7-37)
Lys18-GLP-1 (7-37)
Gly′-GLP-1 (7-36) NH2
Gly′-GLP1 (7-37) OH, Val′-GLP-1 (7-37) OH, Met8-GLP-1 (7-37) OH, acetyl-Lys9-GLP-1 (7-37)
Thr9 GLP-1 (7-37)
D-Thr9-GLP-1 (7-37)
Asn9-GLP-1 (7-37)
D-Asn9-GLP-1 (7-37)
Ser22-Arg23 Arg24-Gln26-GLP-1 (7-37)
Arg23-GLP-1 (7-37)
Arg24-GLP-1 (7-37)
a-methyl-Ala8-GLP-1 (736) NH2
Gly′-Gln2′-GLP-1 (7-37) OH
LY315902, (the activity of which is described in Naslund et al., “Glucagon-like peptide-1 analogue LY315902: effect on intestinal motility and release of insulin and somatostatin”, Regul Pept. 2002 Jun. 15; 106(1-3):89-95)

In another preferred embodiment of the present invention, said GLP-1 molecule is selected from any of the following molecules, which have previously been demonstrated to be capable of activating the GLP-1 receptor and are described by Xiao et al., “Biological Activities of Glucagon-Like Peptide-1 Analogues in Vitro and in Vivo, Biochemistry, 2001, Vol. 40, No. 9, p 2860-2869:
Acetyl-GLP-1
Hexanoic-GLP-1
(dAla2)GLP-1
(Gly2)GLP-1
(Ala4)GLP-1
Hexanoic-(dAla2)GLP-1
Glucagon(1-5)GLP-1
Glucagon(1-10)GLP-1
PACAP(1-5)GLP-1
PACAP(1-10)GLP-1
PHI(1-5)GLP-1
PHI(1-10)GLP-1
Secretin(1-10)GLP-1
VIP(1-5)GLP-1
VIP(1-10)GLP-1
(Ala12/16)GLP-1
(Ala8/11/16)GLP-1
(Ala8/12/16)GLP-1
(dHis1)GLP-1
desaminoHis1 GLP-1
N-me-His1-GLP-1
Alpha-me-His1-GLP-1
Imi-His1-Glp-1
(Ser2)GLP1

Further suitable GLP-1 molecules are those described in any of the following patent applications, hereby incorporated by reference thereto:
WO91/11457
U.S. Pat. No. 5,118,666
WO 2004/094461 (“Human glucagons-like peptide-1 mimics and their use in the treatment of diabetes and related conditions”)
WO 98/08873, “Use of Glucagon-like peptide-1 (GLP-1) or analogs to abolish catabolic changes after surgery”
U.S. Pat. No. 5,545,618
U.S. Pat. No. 5,188,666
U.S. Pat. No. 5,512,549
WO 03/018516 (“Glucagon-like peptide-1 analogs”)
U.S. Pat. No. 5,120,712

### Homologues of GLP-1 Molecules

A homologue of one or more of the sequences specified herein may vary in one or more amino acids as compared to the sequences defined, but is capable of performing the same function, i.e. a homologue may be envisaged as a functional equivalent of a predetermined sequence.

Thus, in one preferred embodiment of the present invention, the GLP-1 molecule is a homologue of any of the molecules disclosed herein, such a homologue of any of the molecules selected from the group consisting of:
GLP-1 (7-36)amide
GLP-1 (7-37)
Liraglutide
Exenatide (Byetta)
Albugon
CJC-1131
zp-10-AVE0010
BIM51077 (Ipsen)
LY315902
LY307161
S 23521

Thus, in one preferred embodiment of the present invention, the GLP-1 molecule is a peptide containing one or more amino acid substitutions, inversion, additions or deletions, compared with a molecule selected from the group consisting of:
GLP-1 (7-36)amide
GLP-1 (7-37)
Liraglutide
Exenatide (Byetta)
Albugon
CJC-1131
zp-10-AVE0010
BIM51077 (Ipsen)
LY315902
LY307161
S 23521

In one embodiment, the number of substitutions, deletions, or additions is 20 amino acids or less, such as 15 amino acids or less, for example 10 amino acids or less, such as 9 amino acids or less, for example 8 amino acids or less such as 7 amino acids or less, for example 6 amino acids or less, such as 5 amino acids or less, for example 4 amino acids or less, such as 3 amino acids or less, for example 2 amino acids or less (such as 1), or any integer in between these amounts. In one aspect of the invention, the substitutions include one or more conservative substitutions, such as 20 or fewer conservative substitutions, for example 18 or fewer, such as 16 or fewer, for example 14 or fewer, such as 12 or fewer, for example 10 or fewer, such as 8 or fewer, for example 6 or fewer, such as 4 or fewer, for example 3 or fewer, such as 2 or fewer conservative substitutions. A “conservative” substitution denotes the replacement of an amino acid residue by another, biologically active similar residue. Examples of conservative substitution include the substitution of one hydrophobic residue, such as isoleucine, valine, leucine or methionine for another, or the substitution of one polar residue for another, such as the substitution of arginine for lysine, glutamic for aspartic acids, or glutamine for asparagine, and the like. The following table lists illustrative, but non-limiting, conservative amino acid substitutions.

| ORIGINAL | EXEMPLARY |
|---|---|
| RESIDUE | SUBSTITUTIONS |
| ALA | SER, THR, VAL, GLY |
| ARG | LYS |
| ASN | HIS, SER |
| ASP | GLU, ASN |
| CYS | SER |
| GLN | ASN, HIS |
| GLU | ASP, GLU |
| GLY | ALA, SER |
| HIS | ASN, GLN |
| ILE | LEU, VAL, THR |
| LEU | ILE, VAL |
| LYS | ARG, GLN, GLU, THR |
| MET | LEU, ILE, VAL |
| PHE | LEU, TYR |
| SER | THR, ALA, ASN |
| THR | SER, ALA |
| TRP | ARG, SER |
| TYR | PHE |
| VAL | ILE, LEU, ALA |
| PRO | ALA |

Other GLP-1 homologues suitable for the uses and methods of the present invention are peptide sequences having greater than 50 percent sequence identity, and preferably greater than 90 percent sequence identity (such as greater than 91% sequence identity, for example greater than 92% sequence identity, such as greater than 93% sequence identity, for example greater than 94% sequence identity, such as greater than 95% sequence identity, for example greater than 96% sequence identity, such as greater than 97% sequence identity, for example greater than 98% sequence identity, such as greater than 99% sequence identity, for example greater than 99.5% sequence identity), to a molecule selected from the group consisting of:
GLP-1 (7-36)amide
GLP-1 (7-37)
Liraglutide
Exenatide (Byetta)
Albugon
CJC-1131
zp-10-AVE0010
BIM51077 (Ipsen)
LY315902
LY307161
S 23521

As used herein, sequence identity refers to a comparison made between two molecules using standard algorithms well known in the art. The preferred algorithm for calculating sequence identity for the present invention is the Smith-Waterman algorithm, where the reference sequence is used to define the percentage identity of polypeptide homologs over its length. The choice of parameter values for matches, mismatches, and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being either a single nucleotide or single amino acid) (Waterman, Bull. Math. Biol. 46, 473-500 (1984)). Insertions and deletions (indels), x, are weighted as

xₖ=1+k/3,

where k is the number of residues in a given insert or deletion (Id.).

For instance, a sequence that is identical to a 42 amino acid residue sequence, except for 18 amino acid substitutions and an insertion of 3 amino acids, would have a percent identity given by:

[(1×42 matches)−(⅓×18 mismatches)−(1+3/3 indels)]/42=81% identity

In one preferred embodiment of the present invention, truncations at the end of the molecule are not taken into account when calculating sequence identity (i.e., if one molecule is longer than the other, only the overlapping lengths of the molecules are used in the sequence identity analysis); in another preferred embodiment of the present invention, truncations are counted as deletions.

A GLP-1 homologue may include the D-amino acid forms and may be a molecule having one or more amino acid substitutions, deletions, inversions, or additions relative to a molecule selected from the group consisting of:
GLP-1 (7-36)amide
GLP-1 (7-37)
Liraglutide
Exenatide (Byetta)
Albugon
CJC-1131
zp-10-AVE0010
BIM51077 (Ipsen)
LY315902
LY307161
S 23521

In one preferred embodiment of the present invention, the GLP-1 molecule is a peptide containing one or more amino acid substitutions, inversion, additions or deletions, compared with GLP-1 (7-36) amide. In one embodiment, the number of substitutions, deletions, or additions is 20 amino acids or less, such as 15 amino acids or less, for example 10 amino acids or less, such as 9 amino acids or less, for example 8 amino acids or less such as 7 amino acids or less, for example 6 amino acids or less, such as 5 amino acids or less, for example 4 amino acids or less, such as 3 amino acids or less, for example 2 amino acids or less (such as 1), or any integer in between these amounts. In one aspect of the invention, the substitutions include one or more conservative substitutions. Examples of suitable conservative substitutions are given above.

Other GLP-1 homologues suitable for the uses and methods of the present invention are peptide sequences having greater than 50 percent sequence identity, and preferably greater than 90 percent sequence identity (such as greater than 91% sequence identity, for example greater than 92% sequence identity, such as greater than 93% sequence identity, for example greater than 94% sequence identity, such as greater than 95% sequence identity, for example greater than 96% sequence identity, such as greater than 97% sequence identity, for example greater than 98% sequence identity, such as greater than 99% sequence identity, for example greater than 99.5% sequence identity), to (1) SEQ ID NOS:1, 2, 3, 4; and/or (2) to truncated sequences thereof. As used herein, sequence identity refers to a comparison made between two molecules using standard algorithms well known in the art. The preferred algorithm for calculating sequence identity for the present invention is the Smith-Waterman algorithm, as described above.

A GLP-1 homologue may also be a molecule having one or more amino acid substitutions, deletions, inversions, or additions relative to human GLP-1 (7-37) and may include the D-amino acid forms.

In another embodiment of the present invention, said homologue of any of the predetermined sequences herein, such as any of SEQ ID NO: 1, 2, 12, or 13, may be defined as:
i) homologues comprising an amino acid sequence capable of binding selectively to the human GLP-1 receptor, and/or
ii) homologues having a substantially similar or higher binding affinity to the GLP-1 receptor than human GLP-1.

In another preferred embodiment, the GLP-1 molecule is an antibody raised against the GLP-1 receptor.

### Chemically Derivatized GLP-1 Molecules

It is further understood that GLP-1 molecules suitable for use in the present invention may be chemically derivatized or altered, for example, peptides with non-natural amino acid residues (e.g., taurine residue, beta- and gamma-amino acid residues and D-amino acid residues), C-terminal functional group modifications, such as amides, esters, and C-terminal ketone modifications and N-terminal functional group modifications, such as acylated amines, Schiff bases, or cyclization, such as found, for example, in the amino acid pyroglutamic acid. One example of a derivatized molecules is Liraglutide.

### Protected GLP-1 Molecules

Furthermore, because the enzyme, dipeptidyl-peptidase IV (DPP IV), may be responsible for the observed rapid in vivo inactivation of administered GLP-1, [see, e.g., Mentlein, R., et al., Eur. J. Biochem., 214:829-835 (1993)], administration of GLP-1 molecules that are protected from the activity of DPP IV may in some embodiments be preferred. Thus, in one preferred embodiment of the present invention, a DPP-IV protected GLP-1 molecule can be used. “DPP-IV protected GLPs” refers to GLP-1 analogs which are resistant to the action of DPP-IV. These include analogs having a modified or D amino acid residue in position 8 and includes biosynthetic GLP-1 analogs having Gly, Val, Thr, Met, Ser, Cys, or Asp in position 8. Other DPP-IV protected GLPs include desamino His derivatives. Preferred embodiments include any of the following: Gly8-GLP-1 (7-36)NH2, Val8-GLP-1 (7-37)OH, a-methyl-Ala8-GL1 (7-36)NH2, and Gly8-Gln21-GLP-1 (7-37)OH, or pharmaceutically acceptable salts thereof.

### GLP-1 Molecule Conjugates

The GLP-1 molecules of the present invention may also be modified with reactive groups capable of forming covalent bonds to yield GLP-1 compounds that are capable of being conjugated to blood components so as to stabilize the GLP-1 molecule. Suitable examples are described in WO 03/103572 (“Modified Glucagon-like peptide-1 analogs”) and US 2004/0138100 (Long lasting synthetic glucagons like peptide (GLP-1).

Thus, in one embodiment of the present invention, the GLP-1 molecule used is a GLP-1 peptide modified with an activated disulfide bond group or S-sulfonate

Other suitable GLP-1 molecules for use in the present invention include those disclosed in WO 00/34331 (“Analogues of GLP-1”), and WO 01/98331 (“Glucagon-like peptide-1 analogs”), the contents of which are incorporated herein by reference.

Further suitable GLP-molecules for use in the present invention are disclosed in WO 00/37098 (“Shelf-stable formulation of Glucagon-like peptide-1”), the contents of which are incorporated by reference herein.

### Particularly Preferred GLP-1 Molecules for Use in the Present Invention:

Particularly preferred GLP-1 molecules for use in the present invention are selected from any of the following:

### Liraglutide

LIRAGLUTIDE (with chemical structure Arg(34)Lys(26)-(N-epsilon-(gamma-Glu(N-alpha-hexadecanoyl))-GLP-1 (7-37)). Preferably, Liraglutide is administered subcutaneously. Preferred doses of Liraglutide are 0.045 mg, 0.225 mg, 0.45 mg, 0.60 mg, or 0.75 mg, such as 0.6 mg. In another preferred embodiment, a dosage of up to 200 ug/kg may be administered, such as up to twice daily, for example 1.25-20 ug/kg*d.

### Exanatide

Exanatide (Amylin Corporation, (see J Biol Chem 1990; 265:20259-62). Exanatide displays similar properties to native GLP-1, and regulates gastric emptying, insulin secretion, food intake, and glucagon secretion.

Exanatide is much more potent than native GLP-1, largely due to its resistance to DPP-IV-mediated inactivation. In contrast to GLP-1 which contains an alanine at position 2, exendin-4 has a position 2 glycine, hence it is not a substrate for DP IV and has a much longer t1/2 (half-life) in vivo. In the methods of the present invention, it is preferred that Exanatide is administered in a dosage of 5 micrograms or 10 micrograms, preferably on a daily basis.

### Albugon and CJC-1131

Albugon (GlaxoSmithKline/Human Genome Sciences) and CJC-1131 (Conjuchem Inc.) are GLP-1-albumin proteins which exploit the long circulating half life of albumin to extend the short duration of action of native GLP-1. Whereas CJC-1131 is a human GLP-1 analogue that forms a covalent bond with human serum albumin following subcutaneous injection of the free CJC-1131 peptide in vivo, Albugon is a recombinant GLP-1-albumin protein produced ex vivo prior to administration of the much larger single recombinant protein in vivo.

Albugon has been shown to inhibit gastric emptying and inhibit food intake in mice following both icv and peripheral administration. (Diabetes. 2004 September; 53(9):2492-500). In one preferred embodiment of the present invention, Albugon is administered in a (preferably daily) dosage of 150-350 ug/kg.

CJC-1131 is a human GLP-1 analogue, modified to be resistant to DPP-IV, with a reactive chemical linker at the carboxy terminal end of the molecule which permits covalent coupling to albumin (Cys 34 residue) following administration in vivo. As albumin exhibits a long circulating half life in vivo, albumin-conjugated drugs should exhibit prolonged action, and delayed clearance, consistent with the known turnover of albumin in human subjects, which is estimated to exhibit a t1/2 of about 15-19 days.

CJC-1131 has the ability to bind and activate the GLP-1 receptor both in vitro and in vivo, and studies in normal and diabetic rodents demonstrate that CJC-1131 exhibits GLP-1-dependent activities (Diabetes. 2003 March; 52(3):751-9). In one embodiment of the present invention, it is preferred that CJC-1131 is administered in a dosage of 2.1-2.6 ug/kg per day. In another preferred embodiment of the present invention, it is preferred that CJC-1131 is administered in a (preferably daily) dosage of 150-350 ug/kg.

Other particularly preferred GLP-1 molecules for use in the present invention include, but are not restricted to, any of the following: zp-10 (see e.g. J Pharmacol Exp Ther. 2003 November; 307(2):490-6), BIM51077 (Ipsen), LY315902 (Regul Pept. 2002 Jun. 15; 106(1-3):89-95.), LY307161 (Eli Lilly), S 23521 (J Endocrinol. 2005 March; 184(3):505-13).

### Functional Activity of GLP-1 Molecule

The GLP-1 molecules useful in the inventive methods and uses described herein are active at the GLP-1 receptor. The GLP-1 molecules can bind to the receptor, and preferably, stimulate receptor activity.

Thus, preferably, the GLP-1 molecule used in the present invention is defined as one or more of the following:
a) comprising an amino acid sequence capable of being recognised by an antibody, said antibody also recognising human GLP-1, and/or
b) comprising an amino acid sequence capable of binding selectively to the human GLP-1 receptor, or
c) a small molecule capable of binding selectively to the human GLP-1 receptor.

The receptor activity can be measured using different techniques such as detecting a change in the intracellular conformation of the receptor, in the G-protein coupled activities, and/or in the intracellular messengers.

One simple measure of the ability of a GLP-1 molecule to activate the GLP-1 receptor is to measure its EC50, i.e. the dose at which the compound is able to activate the signalling of the receptor to half of the maximal effect of the compound. The receptor can either be expressed endogenously on primary cells cultures, for example pituitary cells, or heterologously expressed on cells transfected with the GLP-1 receptor. Whole cell assays or assays using membranes prepared form either of these cell types can be used depending on the type of assay.

GLP-1 biological activity can be determined by standard methods, in general, by receptor-binding activity screening procedures which involve providing appropriate cells that express the GLP-1 receptor on their surface, for example, insulinoma cell lines such as RINmSF cells or INS-1 cells. See also Mojsov, Int J Pept Protein Res 40, 333-43 (1992) and EP0708170A2. Cells that are engineered to express a GLP-1 receptor also can be used. In addition to measuring specific binding of tracer to membrane using radioimmunoassay methods, cAMP activity or glucose dependent insulin production can also be measured. In one method, a polynucleotide encoding the GLP-1 receptor is employed to transfect cells to thereby express the GLP-1 receptor protein. Thus, for example, these methods may be employed for screening for a receptor agonist by contacting such cells with compounds to be screened and determining whether such compounds generate a signal, i.e activate the receptor.

Polyclonal and monoclonal antibodies can be utilized to detect purify and identify GLP-1 like molecules for use in the methods described herein. Antibodies such as ABGA1178 detect intact unprocessed GLP-1 (1-37) or N-terminally-truncated GLP-1 (7-37) or (7-36)amide. Other antibodies detect on the very end of the C-terminus of the precursor molecule, a procedure which allows by subtraction to calculate the amount of biologically active truncated peptide, i.e. GLP-1 (7-37)amide (Orskov et al., Diabetes, 42, 658-661 (1993); Orskov et al., J. Clin. Invest. 87, 415-423 (1991)).

Other screening techniques include the use of cells which express the GLP-1 receptor, for example, transfected CHO cells, in a system which measures extracellular pH or ionic changes caused by receptor activation. For example, potential agonists may be contacted with a cell which expresses the GLP-1 protein receptor and a second messenger response, e.g. signal transduction or ionic or pH changes, may be measured to determine whether the potential agonist is effective.

In one embodiment the binding of a GLP-1 molecule to the GLP-1 receptor can be measured by the use of the assay described herein above.

A GLP-1 molecule for use according to the invention preferably has at least about 50%, at least about 60%, at least about 70%, at least about 80%, or at least about 90%, functional activity relative to human GLP-1 as determined using the assay described herein above, and/or an EC50 greater than about 1,000, greater than about 100, or greater than about 50, or greater than about 10. Greater refers to potency and thus indicates a lesser amount is needed to achieve binding inhibition.

In one embodiment of the invention, the compound has a potency (EC50) on the GLP-1 receptor of less than 500 nM. In another embodiment the compound has a potency (EC50) on the GLP-1 receptor of less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

In a further embodiment the dissociation constant (Kd) of the compound is less than 500 nM. In a still further embodiment the dissociation constant (Kd) of the ligand is less than 100 nM, such as less than 80 nM, for example less than 60 nM, such as less than 40 nM, for example less than 20 nM, such as less than 10 nM, for example less than 5 nM, such as less than 1 nM, for example less than 0.5 nM, such as less than 0.1 nM, for example less than 0.05 nM, such as less than 0.01 nM.

Binding assays can be performed using recombinantly-produced receptor polypeptides present in different environments. Such environments include, for example, cell extracts and purified cell extracts containing the receptor polypeptide expressed from recombinant nucleic acid or naturally occurring nucleic acid; and also include, for example, the use of a purified GLP-1 receptor polypeptide produced by recombinant means or from naturally occurring nucleic acid which is introduced into a different environment.

Using a recombinantly expressed GLP-1 receptor offers several advantages such as the ability to express the receptor in a defined cell system, so that a response to a compound at the receptor can more readily be differentiated from responses at other receptors. For example, the receptor can be expressed in a cell line such as HEK 293, COS 7, and CHO not normally expressing the receptor by an expression vector, wherein the same cell line without the expression vector can act as a control.

### Pharmaceutically Acceptable GLP-1 Salts

A pharmaceutically-acceptable salt form of any of the GLP-1 molecules described herein may be used in the uses and methods of the present invention. Acids commonly employed to form acid addition salts are inorganic acids such as hydrochloric acid, hydrobromic acid, hydroiodic acid, sulfuric acid, phosphoric acid, and the like, and organic acids such as ptoluenesulfonic acid, methanesulfonic acid, oxalic acid, pbromophenyl-sulfonic acid, carbonic acid, succinic acid, citric acid, benzoic acid, acetic acid, and the like.

Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like. Preferred acid addition salts are those formed with mineral acids such as hydrochloric acid and hydrobromic acid, and, especially, hydrochloric acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, and the like. The salt forms are particularly preferred.

A GLP-1 molecule suitable to be used in the present invention may be formulated with one or more excipients before use in the present invention. For example, the active compound used in the present invention may be complexed with a divalent metal cation by well-known methods. Such metal cations include, for example, Zn++, Mn++, Fe++, Co++, Cd++, Ni++, and the like.

Optionally, the active compound used in the present invention may be combined with a pharmaceutically-acceptable buffer, and the pH adjusted to provide acceptable stability, and a pH acceptable for parenteral administration.

Optionally, one or more pharmaceutically-acceptable anti-microbial agents may be added. Meta-cresol and phenol are preferred pharmaceutically-acceptable anti-microbial agents. One or more pharmaceutically-acceptable salts may be added to adjust the ionic strength or tonicity. One or more excipients may be added to further adjust the isotonicity of the formulation. Glycerin is an example of an isotonity adjusting excipient.

### Manufacture of the GLP-1 Molecules for Use in the Present Invention

The GLP-1 molecules suitable for use in the present invention may be manufactured using any suitable method known to one skilled in the art.

For example, the GLP-1 molecules of the invention that are peptides can be made by solid state chemical peptide synthesis. Such peptides can also be made by conventional recombinant techniques using standard procedures described in, for example, Sambrook & Maniatis. “Recombinant”, as used herein, means that a gene is derived from a recombinant (e.g., microbial or mammalian) expression system which has been genetically modified to contain polynucleotide encoding a GLP-1 molecule as described herein. The GLP-1 like peptides can be recovered and purified from recombinant cell cultures by methods including, but not limited to, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (HPLC) can be employed for final purification steps. The GLP-1 molecule peptides of the present invention may be a naturally purified product, or a product of chemical synthetic procedures, or produced by recombinant techniques from prokaryotic or eukaryotic hosts (for example by bacteria, yeast, higher plant, insect and mammalian cells in culture or in vivo). Depending on the host employed in a recombinant production procedure, the polypeptides of the present invention are generally non-glycosylated, but may be glycosylated.

Other suitable methods for preparation of a GLP-1 molecule include the recombinant method described in WO 03/046158 (“A Method for preparing the Recombinant Human Glucagon like peptide-1 (7-37)”), the contents of which are incorporated herein by reference.

The GLP-1 molecule may also be solubilized using e.g. any of the methods described in WO 01/55213 (“Process for solubilizing glucagon-like peptide 1 compounds”), the contents of which are incorporated herein by reference.

### Nausea and Emesis

A common side-effect of some drugs is nausea and emesis. The severity of the symptoms may vary from only mild nausea that may be tolerated by the patient and severe emesis that may lead to malnutrition and even life threatening situation in patients. In certain situations moderate nausea may even be beneficial to the patient for example for patients who are in treatment for obesity, where inhibition of food intake is desired. Side-effects may tempt the patient to withdraw from taking the medication, this may be particularly important for patients who do not suffer from life threatening disease or experience daily symptoms of their disease, such as patients suffering from mild to moderate obesity.

The medicaments may affect each patient differently, and the individuals may experience the side-effects differently. Thus the level of nausea and emesis in the individual patient should be evaluated and the administration aimed at gaining a tolerable level of nausea, including no nausea, while obtaining the desired treatment.

The combination treatment using an anti-emetic drug along with GLP-1 or a GLP-1 analog may, without being bound by theory, be more effective, as higher dosages of the peptide drug may be tolerated and because the patient may be able to have a normal food intake and thereby respond faster to the treatment.

### Anti-Emetic Drugs

In the present context, an “anti-emetic” drug is any drug which counteracts (i.e. reduces or removes) nausea or emesis (vomiting). The experience of nausea and emesis may have many causes and the relief or reduction of the symptoms may be obtained by various mechanisms. The major groups of drugs useful for the treatment of nausea and emesis are; Neuroleptics/anti-psychotics, Antihistamines, Anticholinergic agents, Steroids (corticosteroids), 5HT3-receptor antagonists (serotonin receptor antagonist), NK1-receptor antagonists (Neurokinin I substance P receptor antagonists), Antidopaminergic agents/dopamine receptor antagonists, Benzodiazepines, Cannabinoids. Here below is a non-exhaustive list of members of the different groups of compounds.
1. Neuroleptics/anti-psychotics
   a. dixyrazine
   b. haloperidol
   c. Prochlorperazine (Compazine®)
2. Antihistamines
   a. piperazine derivatives
      i. cyclizine
      ii. meclizine
      iii. cinnarizine
   b. Promethazine
   c. Dimenhydrinate
   d. Diphenhydramine
   e. Hydroxyzine
   f. Buclizine
   g. Meclizine hydrochloride (Bonine, Antivert)
3. Anticholinergic agents (Inhibitors of the acetylcholine receptors.)
   a. Scopolamine
   b. Glycopyrron
   c. Hyoscine
      i. Artane (Trihexy-5 trihexyphenidyl hydrochloride)
      ii. Cogentin (benztropine mesylate)
      iii. Akineton (biperiden hydrochloride)
      iv. Disipal (Norflex orphenadrine citrate)
      v. Kemadrin (procyclidine hydrochloride)
4. Steroids (corticosteroids)
   a. Betamethasone
   b. Dexamethasone
   c. Methylprednisolone
   d. Prednisone®,
   e. Trimethobenzamide (Tigan)
5. 5HT3-receptor antagonists (serotonin receptor antagonist)
   a. Granisetron
   b. Dolasetron
   c. Ondansetron (hydrochloride)
   d. Tropisetron
   e. Ramosetron
   f. Palonosetron
   g. Alosetron
   h. Bemesetron
   i. Zatisetron
   j. Batanopirde
   k. MDL-73147EF;
   l. Metoclopramide
   m. N-3389 (endo-3,9-dimethyl-3,9-diazabicyclo[3,3,1]non-7-yl-1H-indazole-3-carboxamide dihydrochloride),
   n. Y-25130 hydrochloride
   o. MDL 72222
   p. Tropanyl-3,5-dimethylbenzoate
   q. 3-(4-Allylpiperazin-1-yl)-2-quinoxalinecarbonitrile maleate
   r. Zacopride hydrochloride
   s. Mirtazepine (Antidepressant)
6. NK1-receptor antagonists (Neurokinin I substance P receptor antagonists)
   a. Aprepitant
   b. MPC-4505
   c. GW597599
   d. MPC-4505
   e. GR205171 (a selective tachykinin NK1 receptor antagonist)
   f. L-759274
   g. SR 140333
   h. CP-96,345
   i. BIIF 1149, NKP 608C, NKP 608A, CGP 60829, SR 140333 (Nolpitantium besilate/chloride), LY 303870 (Lanepitant), MDL-105172A, MDL-103896, MEN-11149, MEN-11467, DNK 333A, YM-49244, YM-44778, ZM-274773, MEN-10930, S-19752, Neuronorm, YM-35375, DA-5018, MK-869, L-754030, CJ-11974, L-758298, DNK-33A, 6b-l, CJ-11974
   j. Benserazide and carbidopa
   k. TAK-637 [(aR,9R)-7-[3,5-bis(trifluoromethyl)benzyl]-8,9,10,11-tetrahydro-9-methyl-5-(4-methylphenyl)-7H-[1,4]diazocino[2,1-g][1,7]naphthyridine-6,13-dione]
   l. PD 154075 ([(2-benzofuran)-CH₂OCO]-(R)-alpha-MeTrp-(S)-NHCH(CH3) Ph)
   m. FK888, chemical modification of the parent compound, (D-Pro4, D-Trp7,9,10, Phe11)SP4-11.
7. Antidopaminergic agents/dopamine receptor antagonists
   a. Domperidone
   b. Prochlorperazine
   c. Metoclopramide
   d. Chlorpromazine (Thorazine)
   e. Droperidol (Inapsine)
   f. Promethazine (Phenergan)
8. Benzodiazepines(Valium® and others)
9. Non-psychoactive cannabinoids,
   a. Cannabidiol (CBD)
   b. Cannabidiol dimethylheptyl (CBD-DMH)
   c. Tetra-hydro-cannabinol (THC)
   d. Cannabinoid agonists such as WIN 55-212 (a CB1 and CB2 receptor agonist)
   e. Dronabinol (Marinol®)
10. Further cannabinoids
   a. Nabilone (Cesamet)
11. c-9280 (Merck)

In an embodiment the kit of parts according to the invention comprises an anti-emetic drug selected from the group of; neuroleptics, antihistamines, anti-cholinergic agents, steroids, 5HT-3-receptor antagonists, NK1-receptor antagonists, anti-dopaminergic agents/dopamine receptor antagonists, benzodiazepines and non-psychoactive cannabinoids.

In a preferred embodiment the kit of parts comprises an anti-emetic drug that is a 5HT-3-receptor antagonist selected from the group of: Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y-25130 hydrochloride, MDL 72222, Tropanyl-3,5-dimethylbenzoate 3-(4-Allyl-piperazin-1-yl)-2-quinoxalinecarbonitrile maleate, Zacopride hydrochloride and Mirtazepine.

In a more preferred embodiment the kit of parts comprises an anti-emetic drug that is a 5HT-3-receptor antagonist selected from the group of; Granisetron, Dolasetron, Ondansetron hydrochloride, Tropisetron, Ramosetron, Palonosetron, Alosetron, Bemesetron, and Zatisetron.

In a further more preferred embodiment the kit of parts comprises an anti-emetic drug that is a 5HT-3-receptor antagonist selected from the group of; Granisetron, Dolasetron and Ondansetron.

The anti-emetic drug is preferably one which has already been approved by at least one regulatory agency, such as EMEA or FDA, and thus is on the market.

### Indications

Peptide drugs may be administered for the treatment of a variety of diseases or disorders. It is contemplated that the use of an anti-emetic drug may improve GLP-1 or a GLP-1 analog based treatment.

GLP-1 and analogs may for example be used for treatment of the following indications: Obesity, diabetes mellitus^{b} (non-insulin dependent and insulin dependent diabetes mellitus) impaired glucose tolerance, elevated fasting blood glucose levels, abnormal blood glucose levels, metabolic syndrome (Syndrome X), pancreatic P-cell deterioration (including induction/stimulation of P-cell proliferation/replication and differentiate cells into P-cells and inhibit P-cell apoptosis), stroke, myocardial ischaemia or infarction, diseases caused by gastrointestinal hypermotility or dysmotility, and functional gastrointestinal disorder, IBS and Functional Dyspepsia.

For this purpose, treatment and prevention of obesity is meant to encompass regulation (reduction) of food intake, reducing body weight, maintaining body weight (following weight loss or before weight gain has occurred) and preventing and reducing weight gain. Further included is regulation (inhibition/reduction) of appetite, regulation (induction or stimulation) of satiety. Likewise treatment and prevention of diabetes includes treatments of subjects with a partial pancreatectomy, subjects having one or more parents with non-insulin dependent diabetes, subjects who have had gestational diabetes and subjects who have had acute or chronic pancreatitis who are at risk for developing non-insulin dependent diabetes. It further encompasses stimulating insulin gene transcription

The treatment or prevention of the disease, disorder, condition or symptom listed below should be interpreted as including e.g. one or more of:—
a) an amelioration of the symptom(s) associated with the disease, disorder or condition;
b) a delay in the onset of symptoms associated with the disease, disorder or condition;
c) a prevention of the onset of the signs and/or symptoms associated with the disease, disorder or condition;
d) a prevention of the onset of the disease, disorder or condition;
e) a reduction in the severity or frequency of sequelae associated with the disease, disorder or condition;
f) increased longevity and
g) greater quality of life compared with the absence of the treatment,
h) reduced or removed nausea or emesis otherwise experienced as side-effect of the treatment with the peptide drug.

The invention may be used throughout treatment with the peptide drug in question or may be used in those stages of treatment for which the side-effect is observed or where it is desirable to reduce or remove said side-effect. Examples of such stages include
1) an initial treatment stage, e.g. the first days or weeks of treatment until the side-effect is removed or reduced to a tolerable level
2) Stages of treatment where side-effect is more pronounced, e.g. due to the general condition of the patient, use of higher doses of the peptide drug, etc.

Some diseases and disorders subject to treatment with peptide drugs are related to inappropriate uptake or absorption of nutrients and minerals. A subgroup of these peptide drugs may be relevant for the treatment of obesity and diabetes; this includes compounds regulating the function of the gastrointestinal system as well as drugs regulating appetite and food intake.

According to the invention the kit of parts comprises a medicament or medicaments, wherein the medicament(s) is/are for the treatment of a disease of disorders selected from the group of: obesity, diabetes, hypertension and metabolic syndrome.

In a specific embodiment the medicament or medicaments is/are for the treatment of obesity and in a further specific embodiment the medicament or medicaments are for the treatment of diabetes.

In an additional embodiment the medicament or medicaments is/are for the modulation of blood glucose level.

In an additional embodiment the medicament or medicaments is/are for the treatment of stroke and/or myocardial ischaemia or infarction.

In another embodiment the medicament or medicaments is/are for the treatment of functional gastrointestinal disorders, irritable bowel syndrome and functional dyspepsisa.

### Pharmaceutical Compositions

The peptide drug and the anti-emetic drug to be used in the invention are preferably provided in the form of one or more pharmaceutical compositions. It will be understood that the composition will be prepared so as to suit the drug in question as well as its intended administration route and therapeutic use. Below is a description of pharmaceutical compositions useful in the present invention.

Pharmaceutical compositions of the present invention may be prepared by conventional techniques, e.g. as described in Remington: The Science and Practice of Pharmacy 1995, edited by E. W. Martin, Mack Publishing Company, 19th edition, Easton, Pa. The terms “medicament” and “pharmaceutical composition” are used interchangeably herein. The pharmaceutical compositions may appear in conventional forms, for example capsules, tablets, aerosols, solutions, suspensions or topical applications. The term drug is used in its conventional meaning to include the active ingredient in question in any suitable form, such as salt or hydrate form. Accordingly, the term peptide drug is intended to include the peptide as such as well as any suitable salt, hydrate or derivative thereof.

The present invention relates to a medicament comprising GLP-1 or a GLP-1 analog and an anti-emetic drug. While it is possible for GLP-1 or a GLP-1 analog and/or an anti-emetic drug to be administered as the raw chemical(s), it is preferred to administer it in the form of a pharmaceutical composition. The medicaments may be formulated as a combined pharmaceutical preparation or as a separate pharmaceutical preparation. According to the present invention, the medicament(s) comprise(s) GLP-1 or a GLP-1 analog and/or an anti-emetic drug or pharmaceutically acceptable salts, hydrates or derivatives thereof.

The pharmaceutical composition of the present invention preferably comprises a pharmaceutically acceptable carrier, vehicle and/or excipient. The carrier, vehicle and/or excipient should be compatible with the peptide drug, and the anti-emetic drug, respectively and any salt, hydrate or derivative thereof. In a preferred embodiment, the pharmaceutical composition is non immunogenic when administered to a patient in accordance with the present invention.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects such as nausea, dizziness, gastric upset and the like.

The preparation of a pharmaceutical composition that contains active ingredients dissolved or dispersed therein is well understood in the art. Typically such compositions are prepared as sterile injectables either as liquid solutions or suspensions, aqueous or non-aqueous, however, solid forms suitable for solution, or suspensions, in liquid prior to use can also be prepared. The preparation can also be emulsified.

Suitable pharmaceutical carriers include sterile aqueous solution and various organic solvents and inert solid diluents or fillers. Examples of solid carriers are lactose, terra alba, sucrose, cyclodextrin, talc, gelatine, agar, pectin, acacia, magnesium stearate, stearic acid or lower alkyl ethers of cellulose. Examples of liquid carriers are syrup, peanut oil, olive oil, phospholipids, fatty acids, fatty acid amines, polyoxyethylene or water. Suitable excipients are, for example, water, saline, dextrose, glycerol, ethanol or the like and combinations thereof.

In addition, if desired, the composition can contain minor amounts of auxiliary substances such as wetting or emulsifying agents, pH buffering agents and the like which enhance the effectiveness of the active ingredient. Depending on the particular drug in question and its intended delivery route, the formulation may have a pH within the range of 3.5-8, such as in the range 4.5-7.5, such as in the range 5.5-7, such as in the range 6-7.5, such as around 7.3. However, as is understood by one skilled in the art, the pH range may be adjusted according to the drug and to the individual treated and the administration procedure. For example, some peptide drugs may be easily stabilised at a lower pH. Accordingly, in one embodiment of the invention the formulation has a pH within the range 3.5-7, such as 4-6, e.g. 5-6, such as 5.3-5.7, or such as 5.5.

Liquid compositions can also contain liquid phases in addition to or to the exclusion of water. Exemplary of such additional liquid phases are glycerin, vegetable oils such as cottonseed oil, organic esters such as ethyl oleate, and water-oil emulsions.

The pharmaceutical composition of the present invention can include a pharmaceutically acceptable salt of the peptide drug or anti-emetic drug therein. The salt will be one which is acceptable in its therapeutic use. By that it is meant that the salt will retain the biological activity of the peptide drug or anti-emetic drug and the salt will not have untoward or deleterious effects in its application and use in treating diseases.

Pharmaceutically acceptable salts are prepared in a standard manner. If the peptide drug or anti-emetic drug is a base it is treated with an excess of an organic or inorganic acid in a suitable solvent. If the peptide drug or anti-emetic drug is an acid, it is treated with an inorganic or organic base in a suitable solvent.

The pharmaceutically acceptable salt may be an acid addition salts including salts of inorganic acids as well as organic acids. Acid addition salts are formed with free amino groups of the peptide drug/anti-emetic drug. Representative examples of suitable inorganic acids include hydrochloric, hydrobromic, hydriodic, metaphosphoric, phosphoric, sulphuric and nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, ethylenediaminetetraacetic (EDTA), p-aminobenzoic, glutamic, benzenesulfonic and p-toluenesulfonic acids and the like. Further examples of pharmaceutically acceptable inorganic or organic acid addition salts include the pharmaceutical acceptable salts listed in J. Pharm. Sci. 1977, 66, 2, which is incorporated herein by reference. The metal salt may be an alkali metal or earth alkali metal salt. Examples of metal salts include lithium, sodium, potassium and magnesium salts and the like. Examples of ammonium and alkylated ammonium salts include ammonium, methylammonium, dimethylammonium, trimethylammonium, ethylammonium, hydroxyethylammonium, diethylammonium, butylammonium and tetramethylammonium salts and the like.

Salts formed with the free carboxyl groups can be derived from inorganic bases such as, for example, sodium, potassium, ammonium, calcium or ferric hydroxides, and such organic bases as isopropylamine, trimethylamine, 2-ethylamino ethanol, histidine, procaine and the like.

Also included within the scope of pharmaceutical acceptable acid addition salts of GLP-1 or a GLP-1 analog or anti-emetic drug of the invention is any hydrate (hydrated form) thereof.

The pharmaceutical composition of the invention may further comprise transport molecules. Transport molecules are primarily added in order to increase the half-life of the one or more of drugs used in the invention. Transport molecules act by having incorporated into or anchored to it the drug in question.

Any suitable transport molecules known to the skilled person may be used. Examples of transport molecules are those described in the conjugate section. Other preferred examples are liposomes, micelles, and/or microspheres.

Conventional liposomes are typically composed of phospholipids (neutral or negatively charged) and/or cholesterol. The liposomes are vesicular structures based on lipid bilayers surrounding aqueous compartments. They can vary in their physiochemical properties such as size, lipid composition, surface charge and number and fluidity of the phospholipids bilayers. The most frequently used lipids for liposome formation are: 1,2-Dilauroyl-sn-Glycero-3-Phosphocholine (DLPC), 1,2-Dimyristoyl-sn-Glycero-3-Phosphocholine (DMPC), 1,2-Dipalmitoyl-sn-Glycero-3-Phosphocholine (DPPC), 1,2-Distearoyl-sn-Glycero-3-Phosphocholine (DSPC), 1,2-Dioleoyl-sn-Glycero-3-Phosphocholine (DOPC), 1,2-Dimyristoyl-sn-Glycero-3-Phosphoethanolamine (DMPE), 1,2-Dipalmitoyl-sn-Glycero-3-Phosphoethanolamine (DPPE), 1,2-Dioleoyl-sn-Glycero-3-Phosphoethanolamine (DOPE), 1,2-Dimyristoyl-sn-Glycero-3-Phosphate (Monosodium Salt) (DMPA), 1,2-Dipalmitoyl-sn-Glycero-3-Phosphate (Monosodium Salt) (DPPA), 1,2-Dioleoyl-sn-Glycero-3-Phosphate (Monosodium Salt) (DOPA), 1,2-Dimyristoyl-sn-Glycero-3-[Phospho-rac-(1-glycerol)] (Sodium Salt) (DMPG), 1,2-Dipalmitoyl-sn-Glycero-3-[Phospho-rac-(1-glycerol)] (Sodium Salt) (DPPG), 1,2-Dioleoyl-sn-Glycero-3-[Phospho-rac-(1-glycerol)] (Sodium Salt) (DOPG), 1,2-Dimyristoyl-sn-Glycero-3-[Phospho-L-Serine] (Sodium Salt) (DMPS), 1,2-Dipalmitoyl-sn-Glycero-3-[Phospho-L-Serine) (Sodium Salt) (DPPS), 1,2-Dioleoyl-sn-Glycero-3-[Phospho-L-Serine] (Sodium Salt) (DOPS), 1,2-Dioleoyl-sn-Glycero-3-Phosphoethanolamine-N-(glutaryl) (Sodium Salt) and 1,1′,2,2′-Tetramyristoyl Cardiolipin (Ammonium Salt). Formulations composed of DPPC in combination with other lipid or modifiers of liposomes are preferred e.g. in combination with cholesterol and/or phosphatidylcholine.

Long-circulating liposomes are characterized by their ability to extravasate at body sites where the permeability of the vascular wall is increased. The most popular way to produce long circulating liposomes is to attach hydrophilic polymer polyethylene glycol (PEG) covalently to the outer surface of the liposome. Some of the preferred lipids are: 1,2-Dipalmitoyl-sn-Glycero-3-Phosphoethanolamine-N-[Methoxy(Poly-ethylene glycol)-2000] (Ammonium Salt), 1,2-Dipalmitoyl-sn-Glycero-3-Phospho-ethanolamine-N-[Methoxy(Polyethylene glycol)-5000] (Ammonium Salt), 1,2-Dioleoyl-3-Trimethylammonium-Propane (Chloride Salt) (DOTAP).

Possible lipids applicable for liposomes are supplied by Avanti, Polar lipids, Inc, Alabaster, Ala. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

A variety of methods are available for preparing liposomes, as described in, e.g., Szoka et al., Ann. Rev. Biophys. Bioeng. 9:467 (1980), U.S. Pat. Nos. 4,235,871, 4,501,728 and 4,837,028, all of which are incorporated herein by reference. Another method produces multilamellar vesicles of heterogeneous sizes. In this method, the vesicle-forming lipids are dissolved in a suitable organic solvent or solvent system and dried under vacuum or an inert gas to form a thin lipid film. If desired, the film may be redissolved in a suitable solvent, such as tertiary butanol, and then lyophilized to form a more homogeneous lipid mixture which is in a more easily hydrated powderlike form. This film is covered with an aqueous solution of the targeted drug and the targeting component and allowed to hydrate, typically over a 15-60 minute period with agitation. The size distribution of the resulting multilamellar vesicles can be shifted toward smaller sizes by hydrating the lipids under more vigorous agitation conditions or by adding solubilizing detergents such as deoxycholate. Additionally, the liposome suspension may include lipid-protective agents which protect lipids against free-radical and lipid-peroxidative damages on storage. Lipophilic free-radical quenchers, such as alpha-tocopherol and water-soluble iron-specific chelators, such as ferrioxianine, are preferred.

Micelles are formed by surfactants (molecules that contain a hydrophobic portion and one or more ionic or otherwise strongly hydrophilic groups) in aqueous solution. As the concentration of a solid surfactant increases, its monolayers adsorbed at the air/water or glass/water interfaces become so tightly packed that further occupancy requires excessive compression of the surfactant molecules already in the two monolayers. Further increments in the amount of dissolved surfactant beyond that concentration cause amounts equivalent to the new molecules to aggregate into micelles. This process begins at a characteristic concentration called “critical micelle concentration”.

Common surfactants well known to one of skill in the art can be used in the micelles of the present invention. Suitable surfactants include sodium laureate, sodium oleate, sodium lauryl sulfate, octaoxyethylene glycol monododecyl ether, octoxynol 9 and PLURONIC F-127 (Wyandotte Chemicals Corp.). Preferred surfactants are nonionic polyoxyethylene and polyoxypropylene detergents compatible with IV injection such as, TWEEN-80, PLURONIC F-68, n-octyl-beta-D-glucopyranoside, and the like. In addition, phospholipids, such as those described for use in the production of liposomes, may also be used for micelle formation.

In a preferred embodiment of the invention the medicament of the invention comprises GLP-1 or a GLP-1 analog and/or an anti-emetic drug or a salt thereof, as a lyophilisate and a solvent, said lyophilisate and said solvent being in separate compartments until administration. In another embodiment the composition is a solution of the peptide drug and/or anti-emetic drug or a salt thereof. In both embodiments the solvent may be any suitable solvent, such as described herein, and preferably the solvent is saline.

The invention also relates to a method for preparing a medicament or pharmaceutical composition of the invention, comprising admixing at least one peptide drug and/or one anti-emetic drug or a salt thereof according to the present invention with a physiologically acceptable carrier.

In one aspect the invention relates to a pharmaceutical composition comprising GLP-1 or a GLP-1 analog and an anti-emetic drug, or pharmaceutically acceptable salts thereof.

### Formulations

### Buffer and Other Excipients

GLP-1 molecules themselves exhibit a buffering capacity. However, to maintain the pH of the composition for long term storage and stability, it is preferable to add a buffer, such as TRIS. In one preferred embodiment, the formulation has a pH that is about 8.2 to about 8.8, such as about 8.3 to about 8.6, for example about 8.4 to about 8.5. As used in this specification with respect to pH, the term “about” means plus or minus 0.1 pH units. Thus, a pH of “about 8.5” denotes a pH of 8.4 to 8.6. The buffers which are used may be e.g. tromethane (TRIS), and amino-acid based buffers such as lysine and hydroxy-lysine. The term “TRIS” refers to 2-amino-2-hydroxymethyl-1,3-propanediol (also known in the art as tromethane, trimethylol aminomethane or tris (hydroxymethyl)aminomethane), and to any pharmacologically acceptable salt thereof. The free base and the hydrochloride form are two common forms of TRIS.

The concentration of the GLP-1 molecule that is used in the inventive formulation is in one preferred embodiment about 0.30 to about 0.65 mg/ml of the GLP-1 molecule, such as about 0.5 mg/ml of a GLP-1 molecule.

The GLP-1 molecule may also be formulated with a preservative, such as a phenolic preservative, for example m-cresol, phenol, benzyl alcohol, or methyl paraben. One preferred amount of preservative is from about 2 mg/ml to about 6 mg/ml. However, one skilled in the art is aware that the concentration of preservative necessary for effective preservation depends on the preservative used, the pH of the formulation, and whether substances that bind or sequester the preservative are also present. Preferably, m-cresol is used in the formulations as a preservative.

While a buffer and a preservative are most preferably included in the formulation, other additional excipients may be included, such as a tonicity modifier and/or a surfactant as well as distilled water for injections. The tonicity modifier may be included to make the formulation approximately isotonic with bodily fluid depending on the mode of administration. The concentration of the tonicity modifier is in accordance with the known concentration of a tonicity modifier in a peptide formulation. A preferable tonicity modifier used in the present invention is glycerol.

### Administration Forms

The medicament of the invention may be administered by any suitable form, such as one or more of the following administration forms: oral, nasal, parenteral, including subcutaneously, intravenously and intramuscularly, peripherally, topical, buccal, sublingual, transdermal, inhalation, needle-free or in the form of a suppository. When delivered through a mucosal membrane this may be any mucosal membrane of the individual to which the biologically active substance is to be given, e.g. in the nose, vagina, eye, mouth, genital tract, lungs, gastrointestinal tract, or rectum, preferably the mucosa of the nose, mouth or vagina.

Compounds of the invention may preferably be administered parenterally, that is by intravenous, intramuscular, subcutaneous, intranasal, intrarectal, intravaginal or intraperitoneal administration. The subcutaneous and intramuscular forms of parenteral administration are generally most preferred.

Appropriate dosage forms for such administration may be prepared by conventional techniques as described below.

In an embodiment the medicament(s) is/are for peripheral, parenteral or oral administration.

### Compositions for Parenteral Administration

The pharmaceutical composition(s) according to the invention is preferably formulated for parenteral administration, such as via a subcutaneous, intramuscular, intravenous, intranasal, inhalation, intrarectal, intravaginal, buccal, intraperitoneal, intradermal and transdermal administration route.

The peptide drug and/or anti-emetic drug or a salt thereof may be formulated for parenteral administration (e.g., by injection, for example bolus injection or continuous infusion) and may be presented in unit dose form in ampoules, pre-filled syringes, small volume infusion or in multi-dose sealed containers, such as ampoules and vials with an added preservative.

A pharmaceutical composition for parenteral administration may include sterile aqueous and non-aqueous injectable solutions, dispersions, suspensions or emulsions in oily or aqueous vehicles

Aqueous solutions should be suitably buffered if necessary, and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art and example is aqueous polyethylene glycol.

The active ingredient may be sterile powders, granules, and tablets of the kind previously described, obtained by aseptic isolation of sterile solid or by lyophilisation from solution. The active ingredient may be reconstitution before use with a suitable injectable solution, dispersions or vehicle, e.g., sterile, pyrogen-free water prior to use.

Solutions of peptide drug/anti-emetic drug or pharmaceutically acceptable salts thereof can be prepared in water or saline, and optionally mixed with a non-toxic surfactant. Compositions for intravenous or intra-arterial administration may include sterile aqueous solutions that may also contain buffers, liposomes, diluents and other suitable additives.

Examples of oily or non-aqueous carriers, diluents, solvents or vehicles for parental use include propylene glycol, polyethylene glycol, animal, synthetic or vegetable oils, and injectable organic esters, and may contain formulatory agents such as preserving, wetting, emulsifying or suspending, stabilizing and/or dispersing agents. Specific examples of oils useful in such compositions include peanut, soybean, sesame, cottonseed, corn, olive, petrolatum, and mineral. Suitable fatty acids for use in parenteral compositions include oleic acid, stearic acid, and isostearic acid. Suitable organic esters include fatty acid esters such as ethyl oleate and isopropyl myristate.

Suitable soaps for use in parenteral compositions include fatty alkali metal, ammonium, and triethanolamine salts, and suitable detergents include (a) cationic detergents such as, for example, dimethyl dialkyl ammonium halides, and alkyl pyridinium halides; (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylenepolypropylene copolymers, (d) amphoteric detergents such as, for example, alkyl-beta-aminopropionates, and 2-alkyl-imidazoline quaternary ammonium salts, and (e) mixtures thereof.

The parenteral compositions typically will contain from about 0.5 to about 25% by weight of the active ingredient in solution. Preservatives and buffers may be used. In order to minimize or eliminate irritation at the site of injection, such compositions may contain one or more nonionic surfactants having a hydrophile-lipophile balance (HLB) of from about 12 to about 17. The quantity of surfactant in such compositions will typically range from about 5 to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The pharmaceutical dosage forms suitable for injection or infusion can include sterile aqueous solutions or dispersions comprising the active ingredient that are adapted for administration by encapsulation in liposomes. In all cases, the ultimate dosage form must be sterile, fluid and stable under the conditions of manufacture and storage.

Sterile injectable solutions are prepared by incorporating the compound(s) or pharmaceutically acceptable salt(s) thereof in the required amount in the appropriate solvent with various of the other ingredients enumerated above, as required, followed by filter sterilization.

In a preferred embodiment any of the medicament(s) is/are for parenteral administration. In a specific embodiment any of the medicament(s) is/are for subcutaneous administration.

### Compositions for Oral Delivery

Those peptide drugs and anti-emetic drugs capable of remaining biologically active in an individual after oral administration (such as short peptides and small molecules) can be formulated in a wide range of oral administration dosage forms. The pharmaceutical compositions and dosage forms may comprise the active ingredient of the peptide drug and/or anti-emetic drug or its pharmaceutically acceptable salt or a crystal form thereof as the active component. The pharmaceutically acceptable carriers can be either solid or liquid. Solid form preparations include powders, tablets, pills, capsules, cachets, lozenges, and dispersible granules. A solid carrier can be one or more substances which may also act as diluents, flavoring agents, solubilizers, lubricants, suspending agents, binders, preservatives, wetting agents, tablet disintegrating agents, or an encapsulating material.

Preferably, the composition will be about 0.5% to 75% by weight of the peptide drug and/or anti-emetic drug, with the remainder consisting of suitable pharmaceutical excipients. For oral administration, such excipients include pharmaceutical grades of mannitol, lactose, starch, magnesium stearate, sodium saccharine, talcum, cellulose, glucose, gelatin, sucrose, magnesium carbonate, and the like.

In powders, the carrier is a finely divided solid which is a mixture with the finely divided active component. In tablets, the active component is mixed with the carrier having the necessary binding capacity in suitable proportions and compacted in the shape and size desired. The powders and tablets preferably contain 1-70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the composition of the active compound with encapsulating material as carrier providing a capsule in which the active component, with or without carriers, is surrounded by a carrier, which is in association with it. Similarly, cachets and lozenges are included.

Drops according to the present invention may comprise sterile or non-sterile aqueous or oil solutions or suspensions, and may be prepared in accordance with established procedures. The drops may further comprise a bactericidal and/or fungicidal agent. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Other forms suitable for oral administration include toothpaste, gel dentrifrice or chewing gum. Emulsions may be prepared in solutions in aqueous propylene glycol solutions or may contain emulsifying agents such as lecithin, sorbitan monooleate, or acacia. Aqueous solutions can be prepared by dissolving the active component in water and adding suitable colorants, flavors, stabilizing and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, emulsions, syrups and elixirs and may contain, in addition to the active component, colorants, flavors, stabilizers, buffers, artificial and natural sweeteners, dispersants, thickeners, solubilizing agents, and the like.

In an embodiment any or both of the medicament(s) is/are for oral administration. For instance, at least the anti-emetic drug is in the form of a medicament useful for oral administration.

### Compositions for Topical Administration

In one embodiment the medicament(s) may be provided in a form suitable for topical delivery. Regions for topical administration include the skin surface. Compositions for topical administration via the skin and mucous membranes should not give rise to signs of irritation, such as swelling or redness.

The medicament(s) can be administered transdermally. Transdermal administration typically involves the delivery of a medicament for percutaneous passage of the drug into the systemic circulation of the patient. The skin sites include anatomic regions for transdermally administering the drug and include the forearm, abdomen, chest, back, buttock, mastoidal area, and the like.

The medicament(s) may be formulated for topical administration to the epidermis as ointments, creams, gels or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Compositions suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

### Compositions for Aerosol, Nasal or Inhalation Delivery

The medicament(s) may also be administered by inhalation, e.g. by intranasal and oral inhalation administration, e.g. to the respiratory tract. Appropriate dosage forms for such administration, such as an aerosol formulation or a metered dose inhaler, may be prepared by conventional techniques.

The medicament(s) may also be formulated for nasal administration. The solutions or suspensions are applied directly to the nasal cavity by conventional means, for example with a dropper, pipette or spray. The compositions may be provided in a single or multidose form. In the latter case of a dropper or pipette this may be achieved by the patient administering an appropriate, predetermined volume of the solution or suspension. In the case of a spray this may be achieved for example by means of a metering atomizing spray pump. A suitable formulation for nasal administration is described in EP 1 466 610.

For inhalation, the medicament(s) can be formulated in accordance with well known methods, e.g. as an aerosol, a dry powder or solubilized such as in microdroplets, preferably in a device intended for such delivery (such as commercially available from Aradigm, Alkermes or Nektar).

Compositions administered by aerosols may be prepared, for example, as solutions in saline, employing benzyl alcohol or other suitable preservatives, absorption promoters to enhance bioavailability, employing fluorocarbons, and/or employing other solubilizing or dispersing agents in accordance with methods known in the art.

In an embodiment any of the medicaments is/are for nasal administration.

### Composition for Administration as Suppositories

The medicament(s) may also be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The active compound may be formulated into a suppository comprising, for example, about 0.5% to about 50% of a compound of the invention, disposed in a polyethylene glycol (PEG) carrier (e.g., PEG 1000 [96%] and PEG 4000 [4%]).

### Administration

In accordance with the invention the peptide drug and/or anti-emetic drug may be provided by any administration form that allows sufficient levels of the bioactive form of the drugs to reach their respective receptors and thus to ensure robust and appropriate stimulation, without leading to desensitization of the system.

Furthermore, when the peptide drug is to be given for a longer period of time, e.g. for more than one week, one month, or even longer it is preferred that the administration form is well suited for such prolonged treatment.

When the present invention is practised using a combination product comprising GLP-1 or a GLP-1 analog and an anti-emetic drug it follows that the product is preferably administered via the same administration route, i.e. an administration route that is suitable for both drugs. Such administration may e.g. by parenteral, e.g. subcutaneous, or any of the other parenteral administration routes described herein, or may be oral.

When in a combination product, the peptide drug and the anti-emetic drug may be administered simultaneously (i.e. released simultaneously in the body after administration) or sequentially (i.e. released at different points in time after administration). Accordingly, the formulation used for preparing the combination product may be one which ensures simultaneous release of the drugs in the body or sequential release, as appropriate.

When the present invention is practised using the peptide drug and the anti-emetic drug as discrete medicaments the administration form may be different or the same. The administration form for each medicament may be any of those described herein. As an example, the peptide drug may be administered parentally, e.g. subcutaneously, or by inhalation or intranasally, and the anti-emetic drug may be administered orally.

For simultaneous or sequential administration it is preferred that the medicaments are given by the same administration route using a formulation that ensures they be released simultaneously in the body.

Suitable dosing regimens for the medicament(s) of the present invention are preferably determined taking into account factors well known in the art including type of subject being dosed; age, weight, sex and medical condition of the subject; the route of administration; the renal and hepatic function of the subject; the desired effect; and the particular drug. Optimal precision in achieving concentrations of drug within the range that yields efficacy without toxicity requires a regimen based on the kinetics of the drug's availability to target sites. This involves a consideration of the distribution, equilibrium, and elimination of a drug.

### Medicaments

The peptide drug and anti-emetic drug to be used in the present invention may be provided as a kit of parts medicament, or as a combination product, e.g. a product comprising:
i. GLP-1 or a GLP-1 analog and
ii. an anti-emetic drug,
   in a combined medicament and/or in two distinct medicaments for separate, sequential and/or simultaneous administration to a subject.

The product may according to the invention have one or more of the characters of the kit of parts as defined above.

Medicaments are preferably packed in suitable packages. Tablets and soluble tablets are conventionally packed in protective containers such as screw cap bottles, aluminium foil sachets, plastics or metal tubes, or aluminium blister packs. Soluble powders or granules are preferably packed in individual packages such as bags, sacks, sachets or saccules each containing a dose of the pharmaceutical medicament. The bags may be made of water resistant or damp-proof materials, such as aluminium foil. It may be appropriate to incorporate a desiccant in the packages. Solutions, suspensions or emulsions may be packed in sterile vials for single or multiple usages. For parenteral, in particular subcutaneous administration it is may be preferred that the medicament is packed in the form of a cartridge, such as a cartridge for an injection pen, the injection pen being such as an injection pen known from insulin treatment.

When the medical packaging comprises more than one dosage unit, it is preferred that the medical packaging is provided with a mechanism to adjust each administration to one dosage unit only.

### Use

According to the invention the compounds, the peptide drug and/or the anti-emetic drug may be used for the manufacture of medicaments.

One aspect of the invention relates to the use of a composition comprising
i. GLP-1 or a GLP-1 analog and
ii. an anti-emetic drug
   in the manufacturing of a medicament.

The invention further relates to the use of GLP-1 or a GLP-1 analog and/or an anti-emetic drug for the manufacture of a medicament for use in combination therapy; involving separate, sequential or/and simultaneous administration of:
i. a medicament comprising both GLP-1 or a GLP-1 analog and an anti-emetic drug or,
ii. a medicament comprising GLP-1 or a GLP-1 analog and a medicament comprising an anti-emetic drug or,
iii. a medicament comprising both GLP-1 or a GLP-1 analog and an anti-emetic drug, and a separate medicament comprising GLP-1 or a GLP-1 analog or,
iv. a medicament comprising both GLP-1 or a GLP-1 analog and an anti-emetic drug and a separate medicament comprising an anti-emetic drug.

The use further includes the manufacturing of medicaments according to the invention.

### Combination Therapy

Combination therapy involves administration of at least two drugs to a subject. The two drugs may be administered separately and thus be manufactured as distinct medicaments, whereby combination treatment includes the coordinated administration of both drugs within a certain time period, such as with in a week or less.

In an embodiment the invention relates to a medicament comprising an anti-emetic drug and a medicament comprising GLP-1 or a GLP-1 analog for combination treatment involving separate administration.

A further mode of practising combination therapy involves sequential administration, wherein sequential administration prescribes the prior administration of the one drug before administration of the second (and any subsequent) drugs. The administration of the first drug may be administered such as up to a week before administration of the second (and any subsequent drug), or such as up to 24 hours before administration of the second (and any subsequent drug), or such as up to 12 hours before administration of the second (and any subsequent drug), or such as up to 6 hours before administration of the second (and any subsequent drug), or such as up to 3 hours before administration of the second (and any subsequent drug), or such as up to 1 hour before administration of the second (and any subsequent drug), or such as up to 30 minutes before administration of the second (and any subsequent drugs), e.g. up to 25 or 20 minutes prior to administration of the second (and any sub-sequent drug) or such as up to 15 minutes before administration of the second (and any subsequent drug), e.g. up to 10 minutes before, or such as up to 5 minutes before administration of the second (and any subsequent drug).

In another embodiment the invention the peptide drug and the anti-emetic drug are given at different times and frequencies from each other. For instance, the anti-emetic drug may be given once a day, and the peptide drug may be given 1-3 times a day. As an example the anti-emetic drug is given once a day in the morning, and the peptide drug is given 1-3 times a day, e.g. in the morning, at noon and in the evening. The time difference between the anti-emetic drug and the peptide drug may be as defined above.

In an embodiment the invention relates to a medicament comprising an anti-emetic drug and a medicament comprising GLP-1 or a GLP-1 analog for sequential administration.

A third mode of combination therapy is obtained by simultaneous administration of the two or more drugs. Such treatment regimes may be performed by using one combined medicament comprising the two or more drugs.

In an embodiment the invention relates to a medicament comprising an anti-emetic drug and a separate medicament comprising GLP-1 or a GLP-1 analog for simultaneous administration. The invention further relates to a medicament comprising an anti-emetic drug and GLP-1 or a GLP-1 analog, thereby suited for combination therapy. For simultaneous administration it is desired that the peptide drug and the anti-emetic drug are provided in administration forms suitable for simultaneous administration. The two drugs may be formulated together in one formulation to provide simultaneous administration of both drugs. The two drugs may thus both be formulated in a solution suitable for subcutaneous injection, or other parenteral administration forms such as intranasal, inhalated, sublingual, buccal, transdermal, rectal, vaginal. Furthermore, in one embodiment of the combination treatment the drugs are formulated independently but in such a fashion that the drugs are provided to the patient simultaneously. This may be through formulation and storage in two separate chambers in one device suitable for delivery of said medicaments, such as a capsule with two chambers, a pen suitable for injection with two chambers, an inhalation device equipped with two chambers.

It is further understood that combination therapy may involve combinations of separate, sequential and simultaneous administration; thus the medicaments may be administered separately, sequentially and/or simultaneously to a subject.

It is understood that the administration of the peptide drug and the anti-emetic drug is coordinated to obtain maximum efficiency, and that the precise scheme of administration must be determined individually.

### Methods of Treatment

The findings described here in relates to the efficient treatment obtained by administering one or more medicaments comprising GLP-1 or a GLP-1 analog and an anti-emetic drug separately, sequentially or/and simultaneously.

Thus, in one aspect the invention relates to a method of treatment or prevention of a disease or disorder comprising administering to a subject in need thereof one or more medicaments comprising GLP-1 or a GLP-1 analog and an anti-emetic drug separately, sequentially or/and simultaneously.

Preferably, the disease or disorder is selected from the group consisting of obesity, diabetes, hypertension, metabolic syndrome, constipation, osteoporosis, Behcet's disease, Rheumatoid arthritis, cancer and asthma. Alternatively, the medicament(s) is/are used for the modulation of blood glucose level.

The method of treatment involving administration of two or more discrete medicaments may include a period where a different dosage of one or more of the discrete medicament is administered. In certain cases the dosage of one or more medicaments may be decreased after a period, such as after at least 1 day, such as 2 days, 3 days, 5 days, 7 days, 10 days, 14 days or such as after 21 days. A decrease in on a drug may be applicable, but not limited to, cases where a satisfying result is achieved within a period and the following treatment is for maintaining that result.

Also contemplated is a method where an increased dosages is required for a period, such a situation may be, but is not limited to, situations where a degree of habituation/unresponsiveness is experienced, or in situations where the subject is aware of an acute worsening of the symptoms or faces an event that is expected to provoke worsening of the symptoms.

The method of treatment involving administration of two or more discrete medicaments may include a period wherein administration of one or more discrete medicaments is discontinued. According to the present invention the administration of the anti-emetic drug may be discontinued after a period of 3-21 days, such as 4-14 days, such as 7-14 days, or such as 7-10 days.

The administration of the anti-emetic drug may, according to the invention, be discontinued after a period of 1 day, 2 days, 5 days, 7 days, 10 days, 14 days, 18 days or 21 days, where after only the peptide drug is administered.

Thus, a further embodiment relates to the method of treatment according to the invention, wherein administration of the medicament comprising the anti-emetic drug is discontinued after a period of 7 days.

### Dosing Regimes

The dosage requirements will vary with the particular drug composition employed, the route of administration and the particular subject being treated. Ideally, a patient to be treated by the present method will receive a pharmaceutically effective amount of the compound in the maximum tolerated dose, generally no higher than that required before drug resistance develops.

### Illustrative Examples of Dosing Regimes

For methods of use disclosed herein for the compounds, the daily oral dosage regimen will preferably be from about 0.01 μg/kg to about 80 mg/kg of total body weight. Alternatively the dosage may be 0.001-100 pmol/kg. The daily parenteral dosage regimen about 0.01 μg/kg to about 80 mg/kg of total body weight. The daily topical dosage regimen will preferably be from 0.1 mg to 150 mg, administered one to four, preferably two or three times daily. The daily inhalation dosage regimen will preferably be from about 0.01 mg/kg to about 1 mg/kg per day. It will also be recognized by one of skill in the art that the optimal quantity and spacing of individual dosages of a compound or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound or a pharmaceutically acceptable salt thereof given per day for a defined number of days, can be ascertained by those skilled in the art using conventional course of treatment determination tests.

The term “unit dosage form” as used herein refers to physically discrete units suitable as unitary dosages for human and animal subjects, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on individual differences in pharmacokinetics, drug distribution, and metabolism. The “effective level” can be defined, for example, as the blood or tissue level desired in the patient that corresponds to a concentration of one or more compounds according to the invention.

### Non-Limiting Examples of Suitable GLP-1 Dosages

According to the invention GLP-1 or GLP-1 analogs/homologues may be administered in dosages of 0.01 μg/kg-10 mg/kg, alternatively 0.001-1000 pmol/kg, 0.001-100 pmol/kg, 0.1-100 pmol/kg, 0.1-10 pmol/kg, 0.1 pmol-5 pmol/kg, 1.0-100 pmol/kg, 1.0-50 pmol/kg or 10-50 pmol/kg, once or several times weekly, such as once weekly, such as twice weekly, such as three times weekly, such as once daily, such as twice daily, such as three times daily

A unit dosage form may comprise from 100 pmol to 500 nmol, or such from 500 pmol to 100 nmol, or such as from 1 nmol to 500 nmol, or such as from 1 nmol to 250 nmol, or such as from 5 nmol to 250 nmol, or such as from 15 nmol to 200 nmol, or such as from 20 nmol to 150 nmol, or such as from 10 nmol to 100 nmol, or such as from 10 nmol to 50 nmol, or such as from 10 nmol to 20 nmol, or such as from 50 to 125 nmol of GLP-1 or a GLP-1 analog/homologue.

### Non-Limiting Examples of Suitable Exendin-4 Dosages

According to the invention, exendin-4 may be administered in dosages of 1 μg to 150 μg, such as from 5 μg to 140 μg, such as from 5 μg to 70 μg, such as from 5 μg to 20 μg, such as from 5 μg to 10 μg, such as 5 μg, such as 10 μg, such as 20 μg, such as 70 μg or such as 140 μg, once or several times weekly, such as once weekly, such as twice weekly, such as three times weekly, such as once daily, such as twice daily, such as three times daily.

A unit dosage form may comprise from 1 μg to 150 μg, such as from 5 μg to 140 μg, such as from 5 μg to 70 μg, such as from 5 μg to 20 μg, such as from 5 μg to 10 μg, such as 5 μg, such as 10 μg, such as 20 μg, such as 70 μg or such as 140 μg of exendin-4.

### Non-Limiting Examples of Suitable Dosages of Anti-Emetics

Any suitable dosage of an anti-emetic capable of giving the desired anti-emetic effect can be used. Preferably, the dosages of the anti-emetic are given up to three times daily, such as once or twice daily. Examples of suitable dosage ranges of the anti-emetic include from 0.1 to 500 mg, such as 1-125 mg, for example 1-50 mg or 50-100 mg.

A unit dosage form can thus comprise for example from 1 mg to 150 mg, such as from 5 mg to 140 mg, such as from 5 mg to 70 mg, such as from 5 mg to 20 mg, such as from 5 mg to 10 mg, such as 5 mg, such as 10 mg, such as 20 mg, such as 70 mg or such as 140 mg of the anti-emetic.

Dosing of the anti-emetic depends on the nature and properties of the selected compound. Thus, doses of the anti-emetic relevant to use in connection with the present invention include but are not limited to:
8-16 mg once or twice daily, preferably using Ondansetron as the anti-emetic
1-2 mg once or twice daily, preferably using granisetron as the anti-emetic
2.5-5 mg once daily, preferably using tropisetron as the anti-emetic
80-125 mg once daily, preferably using Aprepitant as the anti-emetic

The dose and frequency of dosing will preferably be chosen to match the selected GLP-1 or GLP-1 analog such that the compounds may be administered in combination.

### EXAMPLES

### Example 1

### Suitable Formulation of a GLP-1 Molecule for Use in the Present Invention

Three formulations will be made as follows:

(A) A 21.5 ml aliquot of peptide solution in water will be mixed with 21.5 ml of 0.63% m cresol-3.2% glycerol and the final pH was set to 8.48. The solution will be passed through a 0.2 micron filter. Then aliquots of the solution, containing 0.5 mg/ml peptide in 0.315% m-cresol-1.6% glycerol at pH 8.48, will be pipetted into parenteral vials and stoppered.
(B) A 21.5 ml aliquot of peptide solution in water will be mixed with 21.5 ml of 0.63% m cresol-3.2% glycerol-0.02 molar L-Lysine pH 8.5 and the final pH will be set to 8.48.

The solution will be passed thru a 0.2 micron filter. Then, aliquots of the solution, containing 0.5 mg/ml peptide in 0.315% m-cresol-1.6% glycerol-0.01 molar L-Lysine at pH 8.48, will be pipetted into parenteral vials and stoppered.

(C) A 21.5 ml aliquot of peptide solution in water will be mixed with 21.5 ml of 0.63% m cresol-3.2% glycerol-0.02 molar Tris buffer pH 8.5 and the final pH will be set to 8.50.

The solution will be passed thru a 0.2 micron filter. Aliquots of the solution, containing 0.5 mg/ml peptide in 0.315% m-cresol-1.6% glycerol-0.01 molar tris at pH 8.50, will be pipetted into parenteral vials and stoppered.

Preparation of the GLP-1 Compounds for Use in the Present Invention by Solid Phase t-Boc Chemistry

Approximately 0.5-0.6 grams (0.38-0.45 mmole) Boc Gly-PAM resin will be placed in a standard 60 ml reaction vessel and double couplings will be run on an Applied Biosytems ABI430A peptide synthesizer. The following side-chain protected amino acids (2 mmole cartridges of Boc amino acids) will be obtained from Midwest Biotech (Fishers, Ind.) and used in the synthesis:

Arg-Tosyl (TOS), Asp-delta-cyclohexyl ester (CHXL), Glu-delta-cyclohexyl ester (CHXL), His-benzyloxymethyl (BOM), Lys-2-chlorobenzyloxycarbonyl (2Cl-Z), Met-sulfoxide (O), Ser-O-benzyl ether (OBzl), Thr-O-benzyl ether (OBzl), Trp-formyl (CHO) and Tyr-2-bromobenzyloxycarbonyl (2Br-Z) and Boc Gly PAM resin. Trifluoroacetic acid (TFA), di-isopropylethylamine (DIEA), 0.5 M hydroxybenzotriazole (HOBt) in DMF and 0.5 M dicyclohexylcarbodiimide (DCC) in dichloromethane were purchased from PE-Applied Biosystems (Foster City, Calif.). Dimethylformamide (DMF-Burdick and Jackson) and dichloromethane (DCM-Mallinkrodt) were purchased from Mays Chemical Co. (Indianapolis, Ind.).

Standard double couplings will be run using either symmetric anhydride or HOBt esters, both formed using DCC. A second set of double couplings (without TFA deprotection) will be run at Trp31, Thr13 and Thr11. At the completion of the syntheses, the N-terminal Boc group will be removed and the peptidyl resins treated with 20% piperidine in DMF to deformylate the Trp side chain. After washing with DCM, the resins will be transferred to a TEFLON reaction vessel and dried in vacuo.

For analogs containing Met, an on-the-resin reduction will be done using TFA/10% dimethyl sulfide (DMS)/2% concentrated HCl. Cleavages will be done by attaching the reaction vessels to a HF (hydrofluoric acid) apparatus (Penninsula Laboratories). 1 ml m-cresol per gram/resin will be added and 10 ml HF (purchased from AGA, Indianapolis, Ind.) will be condensed into the pre-cooled vessel. 1 ml DMS per gram resin will be s added when methionine is present. The reactions will be stirred one hour in an ice bath and the HF removed in vacuo. The residues will be suspended in ethyl ether and the solids will be filtered and washed with ether. Each peptide will be extracted into aqueous acetic acid and either freeze dried or loaded directly onto a reverse-phase column.

Purifications will be run on a 2.2×25 cm VYDAC C18 column in buffer A (0.1% Trifluoroacteic acid in water, B: 0.1% TFA in acetonitrile). A gradient of 20% to 90% B will be run on an HPLC (Waters) over 120 minutes at 10 ml/minute while monitoring the UV at 280 nm (4.0 A) and collecting one minute fractions. Appropriate fractions will be combined, frozen and lyophilized. Dried products will be analyzed by HPLC (0.46×15 cm METASIL AQ C18) and MALDI mass spectrometry.

### Preparation of the GLP-1 Compounds of the Present Invention by Solid Phase F-Moc Chemistry

Approximately 114 mg (50 mMole) FMOC Gly WANG resin (purchased from NovaBiochem, LaJolla, Calif.) will be placed in each programmed well of the 96 well reaction block and double couplings will be run on an Advanced ChemTech 396 peptide synthesizer. Analogs with a C-terminal amide will be prepared using 75 mg (50 μmole) Rink Amide AM resin (NovaBiochem, LaJolla, Calif.).

The following FMOC amino acids will be purchased from Advanced ChemTech (Louisville, Ky.), NovaBiochem (La Jolla, Calif.), and Midwest BioTech (Fishers, Ind.): Arg-2,2,4,6,7-pentamethyldihydrobenzofuran-5-sul-fonyl (Pbf), Asn-trityl (Trt), Asp-beta-t-Butyl ester (tBu), Glu-delta-t-butyl ester (tBu), Gln-trityl (Trt), His-trityl (Trt), Lys-t-butyloxycarbonyl (Boc), Ser-t-butyl ether (OtBu), Thr-t-butyl ether (OtBu), Trp-t-butyloxycarbonyl (Boc), Tyr-t-butyl ether (OtBu).

Solvents dimethylformamide (DMF-Burdick and Jackson), N-methylpyrrolidone (NMP-Burdick and Jackson), dichloromethane (DCM-Mallinkrodt) will be purchased from Mays Chemical Co. (Indianapolis, Ind.).

Hydroxybenzotrizole (HOBt), di-isopropylcarbodiimde (DIC), di-isopropylethylamine (DIEA), and piperidine (Pip) will be purchased from Aldrich Chemical Co (Milwaukee, Wis.).

All amino acids will be dissolved in 0.45 M HOBt in NMP and 50 minutes DIC/HOBt activated couplings will be run after 20 minutes deprotection using 20% Pip/DMF. Each resin will be washed with DMF after deprotections and couplings. After the last coupling and deprotection, the peptidyl resins will be washed with DCM and dried in vacuo in the reaction block.

With the reaction/cleavage block assembly in place, 2 ml Reagent K will be added to each well and the cleavage reaction mixed for 2 hours [Reagent K=0.75 gm phenol, 0.5 ml thioanisole, 0.25 ml ethanedithiol, 0.5 ml water per 10 ml trifluoroacetic acid (TFA), all can be purchased from Aldrich Chemical Co., Milwaukee, Wis.]. The TFA filtrates will be added to 40 ml ethyl ether and the precipitants centrifuged 2 minutes at 2000 rpm. The supernatants will be decanted, the pellets re-suspended in 40 ml ether, re-centrifuged, re-decanted, dried under nitrogen and then in vacuo.

0.3-0.6 mg of each product will be dissolved in 1 ml 0.1% TFA/acetonitrile(ACN) and 20 ul was analyzed on HPLC (0.46×15 cm METASIL AQ C18, 1 ml/min, 45 degrees C., 214 nM (0.2 A), A=0.1% TFA, B=0.1% TFA/50% ACN. Gradient=50% B to 90% B over 30 minutes].

Purifications will be run on a 2.2×25 cm VYDAC C18 column in buffer A (0.1% trifluoroacteic acid in water, B: 0.1% TFA in acetonitrile). A gradient of 20% to 90% B will be run on an HPLC (Waters) over 120 minutes at 10 ml/minute while monitoring the UV at 280 nm (4.0 A) and collecting 1 minute fractions. Appropriate fractions will be combined, frozen and lyophilized. Dried products will be analyzed by HPLC (0.46×15 cm METASIL AQ C18) and MALDI mass spectrometry.

### Example 2

### Assay Demonstrating Anti-Emetic Properties

The anti-emetic properties of a drug prepared according to the present invention, will be demonstrated in an animal model of nausea/emesis. Animals, such as rats treated with intraperitoneal injections of cisplatin will be treated with increasing amounts of the test drug. The anti-emetic effects will be measured using variables such as vomiting, kaolin intake and food-intake, which are all affected by cisplatin and can be normalized with anti-emetic agents.

Study outline: 60 rats will be divided into 5 groups of 12 rats, treated as follows:
Group 1: Placebo ip, placebo test drug
Group 2: Cisplatin ip, placebo test drug
Group 3: Cisplatin ip, test drug at X dose
Group 4: Cisplatin ip, test drug at X*5 dose
Group 5: Cisplatin ip, test drug at X*10 dose

The animals will be treated with Cisplatin/placebo at day 0 and daily treatments of the test drug. Measurements of vomiting, kaolin intake and food-intake will be performed daily for 1 week.

### Example 3

### Assay Demonstrating Anti-Diabetic Properties

The anti-diabetic activity of a drug of the present invention will be demonstrated in an animal model of diabetes, such as ob/ob or db/db mice, Streptozotocin-treated rats, or rats or mice with diet-induced obesity and insulin resistance. The ability of the test drug to lower blood glucose, lower HbA1C, and normalize insulin sensitivity as measured for instance with an oral glucose tolerance test will be used as read outs for the anti-diabetic effects.

Study outline: 200 ob/ob mice will be divided into 5 groups of 40 mice, treated as follows:
Group 1: placebo test drug
Group 2: test drug at Y dose
Group 3: test drug at X dose
Group 4: test drug at X*5 dose
Group 5: test drug at X*10 dose

The animals will be treated daily with the test drug for three weeks. An oral glucose tolerance test will be performed on 10 mice prior to initiation of treatment as well as on 10 mice at the end of each week. The blood glucose will be measured at 10 min intervals for an hour post challenge. The mice will be sacrificed by the end of the glucose tolerance test. Daily blood glucose samples will be taken using tail-blood as well.

### Example 4

### Clinical Trial Protocol

The anti-emetic effects of a drug from this invention will be demonstrated in humans. 12 healthy volunteers will be dosed daily with increasing dosages of the test compound (either GLP-1 compound with an anti-emetic or the GLP-1 compound alone). The nausea level will be measured every 10 min during the first 2 hours post dosing and every hour for 6 hours after that. The level will be measured using standard visual analog scales, where the patient indicates on a 10 cm long bar how nauseated they feel. Dosing will be terminated on an individual basis when intolerable nausea has been reached. Subsequently, the same individuals will be treated with increasing doses of the GLP-1 analog part of the kit used in the first part of the study. It is preferred that the dose level reached with the GLP-1 analog without the anti-emetic is lower than the dose levels reached with the anti-emetic.

### Example 5

### Anti-Diabetic Clinical Trial Protocol

The anti-diabetic effects of a kit derived from this invention will be tested in patients with diabetes. In a multicentre, double-blind, parallel-group, double-dummy study the dose-response relationship of the kit's effects on bodyweight and glycaemic control in subjects with Type 2 diabetes will be studied.

METHODS: Subjects with Type 2 diabetes who have previously been treated with an oral anti-diabetic drug monotherapy and have HbA(1c)< or =10% will be enrolled. After a 4-week metformin run-in period, 210 subjects will be randomized to receive the test drug (dosage according to any of the embodiments herein) once daily or continue on metformin 1000 mg b.d. for 12 weeks. The HbA(1c), and the fasting plasma glucose will be measured daily. Body weight will be measured weekly.

1. A product or kit of parts comprising:
i. a GLP-1 molecule and
ii. an anti-emetic drug
wherein the GLP-1 molecule and the anti-emetic drug are provided in a combined medicament or as discrete medicaments.

2. (canceled)

3. The product or kit of parts according to claim 1 comprising:
a) a combined medicament comprising:
i. a GLP-1 molecule and an anti-emetic drug and optionally
b) one or more discrete medicament(s) comprising
i. a GLP-1 molecule and/or
ii. an anti-emetic drug.

4. The product or kit of parts according to claim 1, wherein the kit of parts comprises GLP-1.

5. The product or kit of parts according to claim 1, wherein GLP-1 is selected from the group of consisting of: GLP-1 (7-33), GLP-1 (7-34), GLP-1 (7-35), GLP-1 (7-36), GLP-1 (7-36)amide, and GLP-1 (7-37), or is a fragment, analog, derivative or homologue thereof.

6. The product or kit of parts according to claim 1, wherein the GLP-1 molecule is selected from the group consisting of: GLP-1 (7-36)amide, GLP-1 (7-37), exendin-4, GLP-1 (7-34), GLP-1 (7-35), GLP-1 (7-36), Gln9-GLP-1 (7-37), D-Gln9-GLP-1 (7-37), Thr16-Lys18-GLP-1 (7-37), Lys18-GLP-1 (7-37), GLP-1 (7-34), GLP-1 (7-35), GLP-1 (7-36), GLP-1 (7-36)NH2, Gly8-GLP-1 (7-36)NH2, Gln9-GLP-1 (7-37), D-Gln9-GLP-1 (7-37), acetyl-Lys9-GLP-1 (7-37), Thr9-GLP-1 (7-37), D-Thr9-GLP-1 (7-37), Asn9-GLP-1 (7-37), D-Asn9-GLP-1 (7-37), Ser22-Arg23-Arg24-Gln26-GLP-1 (7-37), Thr16-Lys18-GLP-1 (7-37), Lys18-GLP-1 (7-37), Arg23-GLP-1 (7-37), Arg24-GLP-1 (7-37), GLP-1 (7-34), GLP-1 (7-35), GLP-1 (7-36) NH2, Gln9-GLP-1 (7-37), d-Gln9-GLP1 (7-37), Thr16-Lys18-GLP-1 (7-37), Lys18-GLP-1 (7-37), Gly′-GLP-1 (7-36)NH2, Gly′-GLP1 (7-37) OH, Val′-GLP-1 (7-37) OH, Met8-GLP-1 (7-37) OH, acetyl-Lys9-GLP-1 (7-37), Thr9 GLP-1 (7-37), D-Thr9-GLP-1 (7-37), Asn9-GLP-1 (7-37), D-Asn9-GLP-1 (7-37), Ser22-Arg23 Arg24-Gln26-GLP-1 (7-37), Arg23-GLP-1 (7-37), Arg24-GLP-1 (7-37), a-methyl-Ala8-GLP-1 (736) NH2, Gly′-Gln2′-GLP-1 (7-37) OH and LY315902, or is selected from the group consisting of: GLP-1 (7-36)amide, GLP-1 (7-37), Liraglutide, Exanatide, Albugon, CJC-1131, zp-10, BIM51077 (Ipsen), LY315902, LY307161 (Eli Lilly), and S23521.

7. The product or kit of parts according to claim 1, wherein the GLP-1 molecule is selected from the group of GLP-1 analogs consisting of: Val8-GLP7-37 (SEQ ID NO: 7), Gln9-GLP7-37 (SEQ ID NO: 8), D-Gln9-GLP7-37 (SEQ ID NO: 9), Lys18-GLP7-37 (SEQ ID NO: 10) and Thr16-Lys18-GLP7-37 (SEQ ID NO: 11).

8. The product or kit of parts according to claim 1, wherein the GLP-1 molecule is selected from the group consisting of: NN2211 (liraglutide), CJC-1131, BIM51077, LY315902, LY307161, GTP-010, AVE-10 (ZP10), AC2592 (GLP-1), DAT™: GLP-1, Exendin-4, Exenatide LAR and ZP10.

9. The product or kit of part according to claim 1, wherein the pH of the product is between 4.0 and 9.0.

10. The product or kit of parts according to claim 1, wherein the anti-emetic drug is selected from the group consisting of neuroleptics, antihistamines, anti-cholinergic agents, steroids, 5HT-3-receptor antagonists, NK1-receptor antagonists, antidopaminergic agents/dopamine receptor antagonists, benzodiazepines and non-psychoactive cannabinoids.

11. The product or kit of parts according to claim 10, wherein the anti-emetic drug is a 5HT-3 receptor antagonist.

12. The product or kit of parts according to claim 11, wherein the 5HT-3 receptor antagonist is selected from the group of; Granisetron, Dolasetron, Ondansetron hydrochloride, Tropisetron, Ramosetron, Palonosetron, Alosetron, Bemesetron, Zatisetron, Batanopirde, MDL-73147EF, Metoclopramide, N-3389, Y-25130 hydrochloride, MDL 72222, Tropanyl-3,5-dimethylbenzoate 3-(4-Allylpiperazin-1-yl)-2-quin [symbol] oxalinecarbonitrile maleate, Zacopride hydrochloride and Mirtazepine.

13. The product or kit of parts according to claim 11, wherein the 5HT-3 receptor antagonist is selected from the group consisting of; Granisetron, Dolasetron, Ondansetron hydrochloride, Tropisetron, Ramosetron, Palonosetron, Alosetron, Bemesetron, and Zatisetron.

14. The method of claim 32, which is for the treatment of a disease or disorder selected from the group consisting of: obesity, diabetes, hypertension, metabolic syndrome, stroke, myocardial ischaemia, infarction, functional gastrointestinal disorders, irritable bowel syndrome and functional dyspepsia.

15-16. (canceled)

17. The method of claim 32, which is for the modulation of blood glucose levels.

18-19. (canceled)

20. The method of claim 32 wherein (i) and/or (ii) are administered peripherally.

21. The method of claim 32 wherein (i) and/or (ii) are administered parenterally.

22. The method of claim 32 wherein (i) and/or (ii) are administered subcutaneously.

23. The method of claim 32 wherein (i) and/or (ii) are administered orally.

24. The method of claim 32, wherein at least two medicaments are administered by the same administration mode.

25-31. (canceled)

32. A method of treatment or prevention comprising administering to a subject one or more medicaments comprising,
i. GLP-1 or a GLP-1 analog and
ii. an anti-emetic drug
separately, sequentially or/and simultaneously for the treatment or prevention of a disease selected from the group of: obesity, diabetes mellitus (non-insulin dependent and insulin dependent diabetes mellitus) impaired glucose tolerance, elevated fasting blood glucose levels, abnormal blood glucose levels, metabolic syndrome (Syndrome X), pancreatic P-cell deterioration (including induction/stimulation of P-cell proliferation/replication and differentiate cells into P-cells and inhibit P-cell apoptosis), stroke, myocardial ischaemia or infarction, diseases caused by gastrointestinal hypermotility or dysmotility, functional gastrointestinal disorders, irritable bowel syndrome and functional dyspepsia.

33-36. (canceled)

37. The method according to claim 32, wherein administration of the medicament comprising the anti-emetic drug is discontinued after a period of 7 days.

38. The method according to claim 32, wherein the GLP-1 molecule is administered in an amount of from 1 μg/kg to about 100 mg/kg.

39. The method according to claim 32, wherein the GLP-1 molecule is administered in a dosage of 0.4-2.4 pmol kg⁻¹ min⁻¹.

40. The method according to claim 32, wherein the GLP-1 molecule is administered in a dosage of 150-350 ug/kg.

41. (canceled)

42. The method of claim 32 in which the administration of (i) and (ii) is simultaneous.

43. The method of claim 32 in which the administration of (i) and (ii) is sequential, in either order.
